(19) United States
(12) Patent Application Publication
(10) Pub. No.: US 2004/0241710 A1
(43) Pub. Date: Dec. 2, 2004

(54) Methods of detecting colorectal cancer

(76) Inventors: Kurt C. Gish, Piedmont, CA (US); David H. Mack, Menlo Park, CA (US); Keith E. Wilson, Belmont, CA (US); Sanford David Markowitz, Pepper Pike, OH (US)

Correspondence Address: HOWREY SIMON ARNOLD & WHITE, LLP, C/O M.P. DROSOS, DIRECTOR OF IP ADMINISTRATION, 2941 FAIRVIEW PK, BOX 7, FALLS CHURCH, VA, 22042, US

(21) Appl. No.: 10/702,180

(22) Filed: Nov. 4, 2003

Related U.S. Application Data
(60) Provisional application No. 60/423,960, filed on Nov. 4, 2002.

(51) Int. Cl. C12Q 1/68; G01N 33/574

(52) U.S. Cl. 435/6; 435/7.23

(57) ABSTRACT

The present invention provides a method of detecting colorectal cancer in a human individual. The method comprises detecting one or more colorectal cancer-associated protein in an extracellular biological sample obtained from a human individual, wherein the presence of colorectal cancer-associated protein in said extracellular biological sample indicates colorectal cancer in said human individual. Preferred colorectal cancer-associated protein is CVA7 or CBF9. Also described herein are methods that can be used to screen candidate bioactive agents for the ability to modulate colorectal cancer. Additionally, methods and molecular targets (genes and their products) for therapeutic intervention in colorectal and other cancers are described.

This application claims the benefit of Provisional Application No. 60/423,960, filed Nov. 4, 2002, which is herein incorporated by reference in their entirety.

### RELATED APPLICATIONS

This application is related to PCT U.S.01/28716, filed Sep. 15, 2001, U.S. Ser. No. 60/350,666 filed Nov. 13, 2001, U.S. Ser. No. 10/087,080 filed Feb. 27, 2002, and U.S. Ser. No. 60/282,698 filed Apr. 9, 2001, U.S. Ser. No. 60/372,246filed Apr. 12, 2002 each of which is herein incorporated by reference in their entirety.

### FIELD OF THE INVENTION

The invention relates to methods of detecting antigens associated with colorectal cancer, and to the use of such antigens and their corresponding and nucleic acids for the diagnosis and prognosis evaluation of colorectal cancer. The invention further relates to methods for identifying and using candidate agents and/or targets which modulate colorectal cancer.

### BACKGROUND OF THE INVENTION

Cancer of the colon and/or rectum (referred to as “colorectal cancer”) is significant in Western populations and particularly in the United States. Cancers of the colon and rectum occur in both men and women most commonly after the age of 50, developing as the result of a pathologic transformation of normal colon epithelium to invasive cancer. Recently, a number of genetic alterations have been implicated in colorectal cancer, including mutations in tumor-suppressor genes and proto-oncogenes. Other recent work suggests that mutations in DNA repair genes also are involved in tumorigenesis. For example, inactivating mutations of both alleles of the adenomatous polyposis coli (APC) gene, a tumor suppressor gene, appears to be one of the earliest events in colorectal cancer, and may even be the initiating event. Other genes implicated in colorectal cancer include the CBF9 gene reported in U.S. patent application Ser. No. 60/350,666 filed Nov. 13, 2001, as well as the MCC gene, the p53 gene, the DCC (deleted in colorectal carcinoma) gene and other chromosome 18q genes, and genes in the TGF-β signaling pathway. For a review, see Molecular Biology of Colorectal Cancer, pp. 238-299, in Curr. Probl. Cancer, September/October 1997; see also Willams, Colorectal Cancer (1996); Kinsella & Schofield, Colorectal Cancer: A Scientific Perspective (1993); Colorectal Cancer: Molecular Mechanisms, Premalignant State and its Prevention (Schmiegel & Scholmerich eds., 2000); Colorectal Cancer: New Aspects of Molecular Biology and Their Clinical Applications (Hanski et al., eds 2000); McArdle et al., Colorectal Cancer (2000); Wanebo, Colorectal Cancer (1993); Levin, The American Cancer Society: Colorectal Cancer (1999); Treatment of Hepatic Metastases of Colorectal Cancer (Nordlinger & Jaeck eds., 1993); Management of Colorectal Cancer (Dunitz et al., eds. 1998); Cancer: Principles and Practice of Oncology (Devita et al., eds. 2001); Surgical Oncology: Contemporary Principles and Practice (Kirby et al., eds. 2001); Offit, Clinical Cancer Genetics: Risk Counseling and Management (1997); Radioimmunotherapy of Cancer (Abrams & Fritzberg eds. 2000); Fleming, AJCC Cancer Staging Handbook (1998); Textbook of Radiation Oncology (Leibel & Phillips eds. 2000); and Clinical Oncology (Abeloff et al., eds. 2000).

Early diagnosis of colorectal cancer has been problematic and limited. Methods of diagnosis and prognosis testing are uncomfortable, invasive and require sample biopsy that can be time consuming. As is the case with most cancers early detection is often the key to good prognosis and cure. Therefore what is needed is a quick, convenient and effective method for detecting colorectal cancer while the cancer is still in a stage where the probability of cure is high. Accordingly, provided herein are exactly such methods as are needed for the diagnosis and prognosis determination of colorectal cancer.

### SUMMARY OF THE INVENTION

The present invention provides a method of detecting colorectal cancer in a human individual. The method comprises: (a) determining the amount of one or more colorectal cancer-associated protein in a first extracellular biological sample obtained from a first human individual; and (b) comparing the amount of said one or more colorectal cancer-associated protein in said first extracellular biological sample with the amount of said one or more colorectal cancer-associated protein in an extracellular biological sample obtained from a normal human individual; whereby a higher amount of colorectal cancer-associated protein in said first extracellular biological sample indicates colorectal cancer in said first human individual. In one embodiment, the colorectal cancer-associated protein is CVA7 or CBF9.

In one embodiment, a method of detecting the presence or absence of a colorectal cancer-associated protein in an extracellular biological sample, is provided. The method comprises contacting the biological sample with a binding agent which specifically binds to colorectal cancer-associated proteins selected from the group consisting of CVA7 and CBF9.

In one embodiment the binding agent specifically binds CVA7. In another embodiment the binding agent specifically binds CBF9. In one embodiment, the biological sample is contacted with the binding agent that specifically binds CVA7 and the binding agent that specifically binds CBF9.

In one embodiment the extracellular biological sample is selected from the group consisting of serum, whole blood, plasma, urine, saliva, sputum and cerebrospinal fluid.

In one embodiment the extracellular biological sample is serum.

In one embodiment, the binding agent is an antibody. In another embodiment, the antibody is a monoclonal antibody. In another embodiment the antibody is a polyclonal antibody.

In one embodiment the binding agent is bound to a solid support, which may include, but is not limited to beads, dipsticks, glass, etc. In another embodiment the solid support comprises nitrocellulose. In yet another embodiment, the solid support is a well of a microtiter plate.

In one embodiment, the binding agent is conjugated to a label. In one embodiment the label is radiolabel. In another embodiment the label is a fluorescent label. In another embodiment the label is a detectable enzyme. In one embodiment the detectable enzyme is alkaline phosphatase.

The present invention also provides a kit for detecting the presence or absence of a colorectal cancer-associated protein in an extracellular biological sample, the kit comprising a binding agent which specifically binds to a colorectal cancer-associated protein selected from the group consisting of CVA7 and CBF9 and assay reagents for detecting the presence or absence of the colorectal cancer-associated protein in the extracellular biological sample.

In one embodiment, the binding agent in the kit is labeled. In another embodiment the kit comprises the binding agent that specifically binds CVA7 and the binding agent that specifically binds CBF9.

In one embodiment the binding agent supplied in the kit is an antibody. In another embodiment the antibody in the kit is a monoclonal antibody. In one embodiment the binding agent supplied in the kit is bound to a solid support.

Other aspects of the invention will become apparent to the skilled artisan by the following description of the invention.

### BRIEF DESCRIPTION OF THE DRAWINGS

FIG. 1 shows the CVA expression in colon cancer tissues and normal body atlas.

FIG. 2 shows the CBF9 expression in colon cancer tissues and normal body atlas.

FIG. 3 shows the detection of secreted CBF9 in control medium, Vaco-CBF9 medium, control medium plasma, Vaco-CBF9 plasma, and Vaco-CBF9 RBC.

### DETAILED DESCRIPTION OF THE INVENTION

### Definitions

The term “extracellular biological sample” refers to biological fluids that may be either circulating or non-circulating. Examples of circulating fluid include extracellular fluid comprising the plasma, serum, whole blood, interstitial fluid, as well as transcellular fluid such as cerebrospinal fluid, synovial fluid and pleural fluid. Examples of non-circulating fluids include, but are not limited to urine, saliva, and sputum.

“Binding agent” refers to any substance that binds in a specific manner to another substance. For example, a binding agent may be an antibody that binds specifically to a colorectal cancer-associated CVA7 or CBF9 protein. Similarly a binding agent may be a nucleic acid that is complementary to a colorectal cancer associated CVA7 and/or CBF9 nucleic acid sequence. Alternatively, a binding agent may be a ligand specific for a particular cell surface receptor, or may also be an enzyme that binds a particular substrate. The binding agent may form an attachment that is either covalent or non-covalent, but in most cases the attachment will be non-covalent.

“Specifically binds” means that an association between two molecular units or assemblies is selective. Specificity is judged by the magnitude of an interaction under a defined set of conditions. For example, specific binding occurs when the molecule under consideration is in direct competitive interaction with other such molecules and the other molecules cannot compete successfully with the molecule under consideration for binding of a particular substance.

By “colorectal cancer” refers to a colon and/or rectal tumor or cancer that is classified as Dukes stage A or B as well as metastatic tumors classified as Dukes stage C or D (see, e.g., Cohen et al., Cancer of the Colon, in Cancer: Principles and Practice of Oncology, pp. 1144-1197 (Devita et al., eds., 5^{th} ed. 1997); see also Harrison's Principles of internal Medicinie, pp. 1289-129 (Wilson et al., eds., 12^{th} ed., 1991). “Treatment, monitoring, detection or modulation of colorectal cancer” includes treatment, monitoring, detection, or modulation of colorectal disease in those patients who have colorectal disease (Dukes stage A, B, C or D) in which expression of CVA7 and/or CBF9, is modulated, e.g. increased or decreased, indicating that the subject is more or less likely to progress to metastatic disease than a patient who does not have an increase or decrease in expression of CVA7 and/or CBF9. In Dukes stage A, the tumor has penetrated into, but not through, the bowel wall. In Dukes stage B, the tumor has penetrated through the bowel wall but there is not yet any lymph involvement. In Dukes stage C, the cancer involves regional lymph nodes. In Dukes stage D, there is distant metastasis, e.g., liver, lung, etc.

By the term “recombinant nucleic acid” herein is meant nucleic acid, originally formed in vitro, in general, by the manipulation of nucleic acid by polymerases and endonucleases, in a form not normally found in nature. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

Similarly, a “recombinant protein” is a protein made using recombinant techniques, e.g. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of a colorectal cancer-associated protein from one organism in a different organism or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed below.

In the broadest sense, then, by “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positively charged backbones (Denpcy et al., Proc. Natl. Acad. Sci: U.S. Pat. No. 92:6097 (1995); non-ionic backbones (U.S. Patent Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Patent Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. These modifications of the ribose-phosphate backbone may be done for a variety of reasons, for example to increase the stability and half-life of such molecules in physiological environments or as probes on a biochip.

These nucleic acid analogs and mixtures of naturally occurring nucleic acids and analogs, mixtures of different nucleic acid analogs, and mixtures of naturally occurring nucleic acids and analogs may be made.

Particularly preferred are peptide nucleic acids (PNA) which includes peptide nucleic acid analogs. These backbones are substantially non-ionic under neutral conditions, in contrast to the highly charged phosphodiester backbone of naturally occurring nucleic acids. The nucleic acids may be single stranded or double stranded, as appropriate, or contain portions of both double stranded or single stranded sequence. The depiction of a single strand (“Watson”) also defines the sequence of the complementary strand (“Crick”); thus the sequences described herein also include the complement of the sequence. The nucleic acid may be DNA, genomic and cDNA, RNA or a mixed polymer, where the nucleic acid contains any combination of deoxyribo- and ribo-nucleotides, and combinations of bases, including uracil, adenine, thymine, cytosine, guanine, inosine, xanthine hypoxanthine, isocytosine, isoguanine, etc. As used herein, the term “nucleoside” includes nucleotides, nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

By “substantially complementary” herein is meant that the probes are sufficiently complementary to the target sequences to hybridize under normal reaction conditions, particularly high stringency conditions, as outlined herein.

“Differential expression,” or grammatical equivalents as used herein, refers to both qualitative as well as quantitative differences in the genes' temporal and/or cellular expression patterns within and among the cells. That is, genes may be turned on or turned off in a particular state, relative to another state. A comparison of two or more states can be made. Preferably the change in expression (i.e. upregulation or downregulation) is at least about 50%, more preferably at least about 100%, more preferably at least about 150%, more preferably, at least about 200%, with from 300 to at least 1000% being especially preferred.

As used herein, the terms “colorectal cancer-associated nucleic acid”, “colorectal cancer-associated protein” or “colorectal cancer-associated polynucleotide” or “colorectal cancer-associated transcript” refers to nucleic acid and polypeptide polymorphic variants, alleles, mutants, and interspecies homologs that: (1) have a nucleotide sequence that has greater than about 60% nucleotide sequence identity, 65%, 70%, 75%, 80%, 85%, 90%, preferably 91%, 92%, 93%, 94%, 95%, 96%, 97%, 98% or 99% or greater or greater nucleotide sequence identity, preferably over a region of over a region of at least about 25, 50, 100, 200, 500, 1000, or more nucleotides, to a CVA7 or CBF9 nucleotide sequence of Table 2; (2) bind to antibodies, e.g., polyclonal antibodies, raised against an immunogen comprising an amino acid sequence encoded by the CVA7 or CBF9 nucleotide sequences of Table 2, and conservatively modified variants thereof; (3) specifically hybridize under stringent hybridization conditions to a CVA7 or CBF9 nucleic acid sequence, or the complement and conservatively modified variants thereof or (4) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acidsequence identity, preferably over a region of over a region of at least about 25, 50, 100, 200, 500, 1000, or more amino acids, to an amino acid sequence encoded by a CVA7 or CBF9 nucleotide sequence of Table 2. A polynucleotide or polypeptide sequence is typically from a mammal including, but not limited to, primate, e.g., human; rodent, e.g., rat, mouse, hamster; cow, pig, horse, sheep, or other mammal. A “colorectal cancer-associated polypeptide” and a “colorectal cancer-associated polynucleotide,” include both naturally occurring and recombinant.

Homology in this context means sequence similarity or identity, with identity being preferred. A preferred comparison for homology purposes is to compare the sequence containing sequencing errors to the correct sequence. This homology will be determined using standard techniques known in the art, including, but not limited to, the local homology algorithm of Smith & Waterman, Adv. Appl. Math. 2:482 (1981), by the homology alignment algorithm of Needleman & Wunsch, J. Mol. Biool. 48:443 (1970), by the search for similarity method of Pearson & Lipman, PNAS U.S. Pat. No. 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387-395 (1984), preferably using the default settings, or by inspection.

In one embodiment, the sequences that are used to determine sequence identity or similarity are selected from the CVA7 or CBF9 sequences set forth in Table 2. In one embodiment the sequences utilized herein are the CVA7 and/or CBF9 sequences set forth in Table 2. In another embodiment, the sequences are naturally occurring allelic variants of the CVA7 and/or CBF9 sequences set forth in Table 2. In another embodiment, the sequences are sequence variants as further described herein.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions, as well as naturally occurring, e.g., polymorphic or allelic variants, and man-made variants. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al, eds. 1995 supplement)).

Preferred examples of algorithms that are suitable for determining percent sequence identity and sequence similarity include the BLAST and BLAST 2.0 algorithms, which are described in Altschul et al., Nuc. Acids Res. 25:3389-3402 (1977) and Altschul et al., J. Mol. Biol. 215:403-410 (1990). BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/).

The BLAST algorithm also performs a statistical analysis of the similarity between two sequences (see, e.g., Karlin & Altschul, Proc. Nat'l. Acad. Sci. USA 90:5873-5787 (1993)).

In one embodiment, the colorectal cancer-associated nucleic acids, proteins and antibodies of the invention are labeled. By “labeled” herein is meant that a compound has at least one element, isotope or chemical compound attached to enable the detection of the compound. In general, labels fall into three classes: a) isotopic labels, which may be radioactive or heavy isotopes; b) immune labels, which may be antibodies, enzymatic components, or antigens; and c) colored or fluorescent dyes. The labels may be incorporated into the colorectal cancer-associated nucleic acids, proteins and antibodies at any position. For example, the label should be capable of producing, either directly or indirectly, a detectable signal. The detectable moiety may be a radioisotope, such as ³H, ¹⁴C, ³²P, 35S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. typically the label will be conjugated to the antibody e.g. using a method described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

“Antibody” refers to a polypeptide comprising a framework region from an immunoglobllin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}l by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, such fragments may be synthesized de novo either chemically or by using recombinant DNA methodology. The term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, chemotherapy component, etc.; or (b) the variable region, or a portion thereof, is altered, replaced or exchanged with a variable region having a different or altered antigen specificity.

A “patient” for the purposes of the present invention includes both humans and other animals, particularly mammals, and primates. The methods are applicable to both human therapy and veterinary applications. In the preferred embodiment the patient is a mammal, and in the most preferred embodiment the patient is human.

The present invention provides a method for detecting colorectal cancer by determining the amount of one or more colorectal cancer-associated protein in an extracellular biological sample obtained from a human individual. The method comprises: (a) determining the amount of one or more colorectal cancer-associated protein in a first extracellular biological sample obtained from a first human individual; and (b) comparing the amount of said one or more colorectal cancer-associated protein in said first extracellular biological sample with the amount of said one or more colorectal cancer-associated protein in an extracellular biological sample obtained from a normal human individual; whereby a higher amount of colorectal cancer-associated protein in said first extracellular biological sample indicates colorectal cancer in said first human individual. In one embodiment, the colorectal cancer-associated protein is CVA7 or CBF9.

A detectable amount of CVA7 and CBF9 protein in blood or serum sample from an individual indicates that the individual has colorectal cancer. The method provides a quick, convenient, and efficient method for the early detection of colorectal cancer. In addition, the methods may be used to provide a prognosis evaluation for the presence, progression, or metastasis of colorectal cancer.

The present invention provides nucleic acid and protein sequences of CVA7 and CBF9. These genes are differentially expressed in colorectal cancer, and are herein termed “colorectal cancer-associated sequences”. Table 2 provides the nucleic acid and protein sequences of the CVA7 and CBF9 genes as well as the Unigene and Exemplar accession numbers for CVA7 and CBF9.

CBF9 has domains that suggest protein interactions. Without wishing to be bound by theory, perhaps partners may exist as blocking access to epitopes or deletional markers for cancer.

In one embodiment, the colorectal cancer-associated CVA7 and CBF9 sequences are from humans; however, colorectal cancer sequences from other organisms may be useful in animal models of disease and drug evaluation or veterinary applications; thus, other colorectal cancer sequences are similarly available, from vertebrates, including mammals, including rodents (rats, mice, hamsters, guinea pigs, etc.), primates, farm animals (including sheep, goats, pigs, cows, horses, etc). Colorectal cancer sequences from other organisms may be obtained using the techniques outlined below.

Colorectal cancer-associated CVA7 and CBF9 sequences can include both nucleic acid and amino acid sequences. In another embodiment, the colorectal cancer-associated sequences are amino acid sequences. In another embodiment the colorectal cancer-associated sequences are nucleic acid sequences.

A colorectal cancer-associated sequence can be initially identified by substantial nucleic acid and/or amino acid sequence homology to the CVA7 and CBF9 colorectal cancer-associated sequences provided herein. Such homology can be based upon the overall nucleic acid or amino acid sequence, and is generally determined as outlined below, using either homology programs or hybridization conditions.

The nucleic acid sequences of the invention can be used to generate protein sequences, e.g. cloning the entire gene and verifying its frame and amino acid sequence, or by comparing it to known sequences to search for homology to provide a frame, assuming the colorectal cancer-associated protein has homology to some protein in the database being used.

The present invention provides colorectal cancer-associated protein sequences. “Protein” in this sense includes proteins, polypeptides, and peptides, terms that are often used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

In one embodiment, the colorectal cancer-associated proteins are secreted or released proteins; the release of which can be either constitutive or regulated. These proteins may have a signal peptide or signal sequence that targets the molecule to the secretory pathway. Secreted proteins are involved in numerous physiological events; by virtue of their circulating nature, they often serve to transmit signals to various other cell types. The secreted protein may function in an autocrine manner (acting on the cell that secreted the factor), a paracrine manner (acting on cells in close proximity to the cell that secreted the factor) or an endocrine manner (acting on cells at a distance). Thus, secreted molecules find use in modulating or altering numerous aspects of physiology. Other soluble proteins may have functions related to extracellular functions, e.g. enzymes, or extracellular metabolic processes. Alternatively, their solubility may be indicative of a physiological abnormality. Colorectal cancer-associated proteins that are soluble proteins are particularly preferred in the present invention as they serve as good targets for diagnostic markers, for example for blood, stool, or serum tests.

In one aspect, the expression levels of CVA7 and/or CBF9 genes are determined in different patient samples for which either diagnosis or prognosis information is desired, to determine whether or not a particular individual has colorectal cancer. Healthy individuals may be distinguished from individuals with colorectal cancer, and among those individuals with colorectal cancer, different prognosis states (good or poor long term survival prospects, for example) may be determined.

Bioinformatics analysis of both CVA7 and CBF9 sequences predicts that these genes encode secreted proteins. Both proteins contain predicted signal sequences. CBF9 also contains von Willebrand factor (VWF) type A domains and epidermal growth factor (EGF) domains. Both of these domains are often found in secreted growth factors. Applicants have discovered that both CBF9 and CVA7 are secreted.

The colorectal cancer-associated sequences of the invention can be identified as follows. Samples of serum or blood are collected from a patient. The samples are treated to extract total protein, or in some cases mRNA may be isolated. Methods for mRNA and protein isolation are known in the art. The CVA7 and CBF9 proteins can then be detected in a total protein preparation using CVA7 or CBF9 specific antibodies, or other methods known in the art. Expression data for the CVA7 and/or CBF9 proteins are thereby generated, and analysis of the data can be scrutinized to so as to provide a colorectal cancer diagnosis, or alternatively, may also be used for prognosis evaluation of an individual with colorectal cancer.

Although CVA7 and/or CBF9 expression may be detected and compared between different individuals by evaluation at the gene transcript, or the protein level, evaluation at the protein level is preferred. To quantify the expression levels of CVA7 and or CBF9, protein expression can be monitored, for example through the use of antibodies to the colorectal cancer-associated CVA7 and/or CBF9 proteins. Standard immunoassays such as ELISAs, etc., or other techniques, including mass spectroscopy assays, 2D gel electrophoresis assays, are all methods contemplated by the invention for the detection of CVA7 and/or CBF9 proteins in patient samples.

In another embodiment, the CVA7 and CBF9 colorectal cancer-associated sequences are up-regulated in colorectal cancer; that is, the expression of these genes is higher in individuals with colorectal carcinoma as compared to healthy individuals. “Up-regulation” as used herein means at least about a 1.1 fold change, preferably a 1.5 or two fold change, preferably at least about a three fold change, with at least about five-fold or higher being preferred.

The present invention provides novel methods for diagnosis and prognosis evaluation for colon cancer, as well as methods for screening for compositions which modulate colon cancer and compositions which bind to modulators of colon cancer. In one aspect, the expression levels of genes are determined in different patient samples for which either diagnosis or prognosis information is desired, to provide expression profiles. An expression profile of a particular sample is essentially a “fingerprint” of the state of the sample; while two states may have any particular gene similarly expressed, the evaluation of a number of genes simultaneously allows the generation of a gene expression profile that is unique to the state of the cell. That is, normal tissue may be distinguished from colon cancer tissue, and within colon cancer tissue, different prognosis states (good or poor long term survival prospects, for example) may be determined. By comparing expression profiles of colon cancer tissue in different states, information regarding which genes are important (including both up- and down-regulation of genes) in each of these states is obtained. The identification of sequences that are differentially expressed in colon cancer tissue versus normal colon tissue, as well as differential expression resulting in different prognostic outcomes, allows the use of this information in a number of ways. For example, the evaluation of a particular treatment regime may be evaluated: does a chemotherapeutic drug act to improve the long-term prognosis in a particular patient. Similarly, diagnosis may be done or confirmed by comparing patient samples with the known expression profiles. Furthermore, these gene expression profiles (or individual genes) allow screening of drug candidates with an eye to mimicking or altering a particular expression profile; for example, screening can be done for drugs that suppress the colon cancer expression profile or convert a poor prognosis profile to a better prognosis profile. This may be done by making biochips comprising sets of the important colon cancer genes, which can then be used in these screens. These methods can also be done on the protein basis; that is, protein expression levels of the colon cancer proteins can be evaluated for diagnostic and prognostic purposes or to screen candidate agents. In addition, the colon cancer nucleic acid sequences can be administered for gene therapy purposes, including the administration of antisense nucleic acids, or the colon cancer proteins (including antibodies and other modulators thereof) administered as therapeutic drugs.

By comparing the expression of CVA7 and CBF9 in individuals experiencing different states of health, information regarding up- and down-regulation of CVA7 and CBF9 in each of these states is obtained. Diagnosis may then be done or confirmed. For example, does a particular patient have the CVA7 or CBF9 gene expression profile of a healthy individual or an individual with colorectal cancer. Alternatively, one may evaluate the data to determine the likely prognosis for an individual with colorectal cancer. In some circumstances the diagnosis may involve determination of other genes in addition to CVA7 and CBF9.

Preparation of CVA7 and CBF9 Specific Antibodies

A. Cloning

To prepare antibodies for the serum detection of CVA7 and CBF9, mRNA is isolated from total cellular RNA by known methods. Once total RNA is isolated, mRNA is isolated by making use of the adenine nucleotide residues known as a poly (A) tail which is found on virtually every eukaryotic mRNA molecule at the 3′ end thereof. Oligonucleotides composed of only deoxythymidine [olgo(dT)] are linked to cellulose and the oligo(dT)-cellulose packed into small columns. When a preparation of total cellular RNA is passed through such a column, the mRNA molecules bind to the oligo(dT) by the poly (A) tails while the rest of the RNA flows through the column. The bound mRNAs are then eluted from the column and collected.

The CVA7 and CBF9 colorectal cancer-associated sequences are initially identified by substantial nucleic acid and/or amino acid sequence homology to the CVA7 and CBF9 colorectal cancer-associated sequences provided herein. Such homology can be based upon the overall nucleic acid or amino acid sequence, and is generally determined as outlined below, using either homology programs or hybridization conditions.

Nucleic acid homology can be determined through hybridization studies. For example, nucleic acids that hybridize under high stringency to the nucleic acid sequences which encode the CVA7 and/or CBF9 peptides identified in Table 2, or their complements, are considered a colorectal cancer-associated sequence. High stringency conditions are known; see for example Maniatis et al., Molecular Cloning: A Laboratory Manual, 2d Edition, 1989, and Short Protocols in Molecular Biology, ed. Ausubel, et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993).

In one embodiment, less stringent hybridization conditions are used; for example, moderate or low stringency conditions may be used, as are known in the art; see Maniatis and Ausubel, supra, and Tijssen, supra.

For selective or specific hybridization, a positive signal is typically at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions.

In addition to hybridization techniques substantial identity between two nucleic acid sequences is indicated when the polypeptide encoded by a first nucleic acid is immunologically cross-reactive with the antibodies raised against the polypeptide encoded by a second nucleic acid. Thus, a polypeptide is typically substantially identical to a second polypeptide, e.g., where the two peptides differ only by conservative substitutions.

Yet another indication that two nucleic acid sequences are substantially identical is that the same primers can be used to amplify the sequences. For polymerase chain reaction (PCR), a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions are readily found in the art. In particular, protocols and guidelines for low and high stringency amplification reactions are provided, e.g., in Innis et al., PCR Protocols, A Guide to Methods and Applications (1990).

B. Expression of Cloned CVA7 and CBF9 Genes

In one embodiment, colorectal cancer-associated nucleic acids encoding the CVA7 and CBF9 colorectal cancer-associated proteins are used to make a variety of expression vectors to express colorectal cancer-associated proteins which can then be used in diagnostic and prognostic assays, as described below. The expression vectors may be either self-replicating extrachromosomal vectors or vectors which integrate into a host genome. Generally, these expression vectors include transcriptional and translational regulatory nucleic acid operably linked to the nucleic acid encoding the colorectal cancer-associated protein. The term “control sequences” refers to DNA sequences necessary for the expression of an operably linked coding sequence in a particular host organism. The control sequences that are suitable for prokaryotes, e.g., include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous.

The transcriptional and translational regulatory nucleic acid will generally be appropriate to the host cell used to express the colorectal cancer-associated protein; e.g., transcriptional and translational regulatory nucleic acid sequences from Bacillus are preferably used to express the colorectal cancer-associated protein in Bacillus. Numerous types of appropriate expression vectors, and suitable regulatory sequences are known for a variety of host cells.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, an expression vector may comprise additional elements. For example, an expression vector may have two replication systems, thus allowing it to be maintained in two organisms, e.g., in mammalian or insect cells for expression and in a procaryotic host for cloning and replication. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In addition, in another embodiment, the expression vector contains a selectable marker gene to allow the selection of transformed host cells. Selection genes are well known and will vary with the host cell used.

The colorectal cancer-associated proteins of the present invention are readily produced by culturing a host cell transformed with an expression vector containing nucleic acid encoding a colorectal cancer-associated protein, under the appropriate conditions to induce or cause expression of the colorectal cancer-associated protein. The conditions appropriate for colorectal cancer-associated protein expression will vary with the choice of the expression vector and the host cell, and will be easily ascertained by one skilled in the art through routine experimentation.

Appropriate host cells include yeast, bacteria, archaebacteria, fungi, and insect and animal cells, including mammalian cells. Of particular interest are E. coli, Sf9 cells, C129 cells, 293 cells, BHK, CHO, COS, HeLa cells, THP1 cell line (a macrophage cell line) and human cells and cell lines.

In one embodiment, the colorectal cancer-associated proteins are expressed in mammalian cells. Mammalian expression systems are also known in the art, and include retroviral systems see e.g., “Expression of Recombinant Genes in Eukaryotic Systems” Abelson et al. eds. (1999) Methods in Enzymology Vol. 306. A preferred expression vector system is a retroviral vector system such as is generally described in PCT/US97/01019 and PCT/US97/01048, both of which are hereby expressly incorporated by reference. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter. Typically, transcription termination and polyadenylation sequences recognized by mammalian cells are regulatory regions located 3′ to the translation stop codon and thus, together with the promoter elements, flank the coding sequence. Examples of transcription terminator and polyadenlytion signals include those derived form SV40.

Methods of introducing exogenous nucleic acid into mammalian hosts, as well as other hosts, are well known, and will depend upon the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In one embodiment, colorectal cancer-associated proteins are expressed in bacterial systems. Bacterial expression systems are well known in the art. Promoters from bacteriophage may also be used and are known in the art. In addition, synthetic promoters and hybrid promoters are also useful; e.g., the tac promoter is a hybrid of the trp and lac promoter sequences. Furthermore, a bacterial promoter can include naturally occurring promoters of non-bacterial origin that have the ability to bind bacterial RNA polymerase and initiate transcription. In addition to a functioning promoter sequence, an efficient ribosome binding site is desirable. The expression vector may also include a signal peptide sequence that provides for secretion of the colorectal cancer-associated protein in bacteria. The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways. These components may be assembled into bacterial expression vectors.

In one embodiment, colorectal cancer-associated proteins are produced in insect cells. Expression vectors for the transformation of insect cells, and in particular, baculovirus-based expression vectors, are available.

In another embodiment, colorectal cancer-associated protein is produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica.

The colorectal cancer-associated protein may also be made as a fusion protein, using available techniques. Thus, for example, for the creation of monoclonal antibodies, if the desired epitope is small, the colorectal cancer-associated protein may be fused to a carrier protein to form an immunogen. Alternatively, the colorectal cancer-associated protein may be made as a fusion protein to increase expression, or for other reasons. For example, for a colorectal cancer-associated peptide, the nucleic acid encoding the peptide may be linked to other nucleic acid for expression purposes.

In addition, as is outlined herein, colorectal cancer-associated proteins can be made that are longer than the CVA7 and CBF9 depicted in Table 2 e.g., by the elucidation of additional sequences, the addition of epitope or purification tags, the addition of other fusion sequences, etc.

In one embodiment, the colorectal cancer-associated protein is purified or isolated after expression. Colorectal cancer-associated proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the colorectal cancer-associated protein may be purified using a standard anti-colorectal cancer antibody column. Mitrafiltration and diafiltration techniques, in conjunction with protein concentration, are also useful. For general guidance in suitable purification techniques, see e.g., Scopes, R., Protein Purification, Springer-Verlag, NY (1982). The degree of purification necessary will vary depending on the use of the colorectal cancer-associated protein. In some instances little or no purification will be necessary.

Colorectal cancer-associated CVA7 and CBF9 proteins of the present invention may be shorter or longer than the wild type amino acid sequences. Thus, in one embodiment, included within the definition of colorectal cancer-associated proteins are portions or fragments of the wild type sequences. In addition, as outlined above, the colorectal cancer-associated nucleic acids of the invention may be used to obtain additional coding regions, and thus additional protein sequence, using techniques known in the art.

In another embodiment, the colorectal cancer-associated proteins are derivative or variant colorectal cancer-associated proteins as compared to the wild-type sequence. That is, as outlined more fully below, the derivative colorectal cancer-associated peptide will contain at least one amino acid substitution, deletion or insertion, with amino acid substitutions being particularly preferred. The amino acid substitution, insertion or deletion may occur at any residue within the colorectal cancer-associated peptide.

Also included in an embodiment of colorectal cancer-associated proteins of the present invention are amino acid sequence variants. These variants typically fall into one or more of three classes: substitutional, insertional or deletional variants. These variants ordinarily are prepared by site specific mutagenesis of nucleotides in the DNA encoding the colorectal cancer-associated protein, using cassette or PCR mutagenesis or other common techniques, to produce DNA encoding the variant, and thereafter expressing the DNA in recombinant cell culture as outlined above. However, variant colorectal cancer-associated protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the colorectal cancer-associated protein amino acid sequence.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the colorectal cancer-associated protein are desired, substitutions are generally made in accordance with the following Table 1:

TABLE 1

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Table 1. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

The variants typically will elicit the same immune response as the naturally-occurring analogue, although variants also are selected to modify the characteristics of the colorectal cancer-associated proteins as needed. Alternatively, the variant may be designed such that the biological activity of the colorectal cancer-associated protein is altered. For example, glycosylation sites may be altered or removed.

C. Raising Antibodies to CVA7 and CBF9 Proteins

Once expressed, and purified if necessary, the CVA7 and CBF9 colorectal cancer-associated proteins are useful in a number of applications.

In one embodiment, the colorectal cancer-associated proteins of the present invention may be used to generate polyclonal and monoclonal antibodies to colorectal cancer-associated proteins, which are useful as described herein. Similarly, the colorectal cancer-associated proteins can be coupled, using standard technology, to affinity chromatography columns. These columns may then be used to purify colorectal cancer antibodies. In another embodiment, the antibodies are generated to epitopes unique to the CVA7 and CBF9 colorectal cancer-associated proteins; that is, the antibodies show little or no cross-reactivity to other proteins.

In one embodiment, when the colorectal cancer-associated protein is to be used to generate antibodies, the colorectal cancer-associated protein should share at least one epitope or determinant with the full length protein. By “epitope” or “determinant” herein is meant a portion of a protein which will generate and/or bind an antibody or T-cell receptor in the context of MHC. Thus, in most instances, antibodies made to a smaller colorectal cancer-associated protein will be able to bind to the full length protein. In one embodiment, the epitope is unique; that is, antibodies generated to a unique epitope show little or no cross-reactivity. In another embodiment, the epitope is selected from a peptide encoded by a nucleic acid of Table 2. In another preferred embodiment, the epitope is selected from the CVA7 and/or CBF9 peptide sequences.

For preparation of antibodies, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3_{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express antibodies (see, eg., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980 , WO 91/00360; WO 92/200373; and EP 03089).

Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the CVA7 or the CBF9 peptide of Table 2, or a peptide encoded by the CVA7 or CBF9 nucleic acids of Table 2 or fragment thereof or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thymoglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include the CBF9 polypeptide or a peptide encoded by a CVA7 and/or CBF9 nucleic acid of Table 2 or a fragment thereof or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

The CVA7 and CBF9 colorectal cancer antibodies of the invention specifically bind to colorectal cancer-associated proteins. By “specifically bind” herein is meant that the antibodies bind to the protein with a binding constant in the range of at least 10⁻⁴-10⁻⁶ M⁻¹, with a preferred range being 10⁻⁷-10⁻⁹M^{-l}. Preferred antibodies will exhibit both high affinity and high selectivity. One can screen for which exhibit low cross reactivity to other proteins e.g., serum or other samples being diagnosed. For ELISA antibodies can be selected that recognize two epitopes for sandwich assay.

In one embodiment the CVA7 and/or CBF9 colorectal cancer-associated proteins against which antibodies are raised are secreted proteins.

Covalent modifications of colorectal cancer-associated polypeptides are included within the scope of this invention. One type of covalent modification includes reacting targeted amino acid residues of a colorectal cancer-associated polypeptide with an organic derivatizing agent that is capable of reacting with selected side chains or the N-or C-terminal residues of a colorectal cancer-associated polypeptide. Derivatization with bifunctional agents is useful, for instance, for crosslinking colorectal cancer-associated sequences to a water-insoluble support matrix or surface for use in the method for purifying anti-colorectal cancer antibodies or screening assays, as is more fully described below. Commonly used crosslinking agents include, e.g., 1,1-bis(diazo-acetyl)-2-phenylethane, glutaraldehyde, N-hydroxy-succinimide esters, for example, esters with 4-azido-salicylic acid, homobifunctional imidoesters, including disuccinimidyl esters such as 3,3′-dithiobis-(succinimidyl-propionate), bifunctional maleimides such as bis-N-maleimido-1,8-octane and agents such as methyl-3-[(p-azidophenyl)-dithio]pro-pioimi-date.

Other modifications include deamidation of glutaminyl and asparaginyl residues to the corresponding glutamyl and aspartyl residues, respectively, hydroxylation of proline and lysine, phosphorylation of hydroxyl groups of seryl, threonyl or tyrosyl residues, methylation of the α-amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group.

Another type of covalent modification of the colorectal cancer-associated polypeptide included within the scope of this invention comprises altering the native glycosylation pattern of the polypeptide. “Altering the native glycosylation pattern” is intended for purposes herein to mean deleting one or more carbohydrate moieties found in native sequence colorectal cancer-associated polypeptide, and/or adding one or more glycosylation sites that are not present in the native sequence colorectal cancer-associated polypeptide.

Addition of glycosylation sites to colorectal cancer-associated polypeptides may be accomplished by altering the amino acid sequence thereof. The alteration may be made, for example, by the addition of, or substitution by, one or more serine or threonine residues to the native sequence colorectal cancer-associated polypeptide (for O-linked glycosylation sites). The colorectal cancer-associated amino acid sequence may optionally be altered through changes at the DNA level, particularly by mutating the DNA encoding the colorectal cancer-associated polypeptide at preselected bases such that codons are generated that will translate into the desired amino acids.

Detection of CVA7 and CBF9 in Biological Samples

In a most preferred embodiment, antibodies find use in diagnosing colorectal cancer proteins may be found in circulating or non-circulating body fluids. Blood samples are convenient samples to be probed or tested for the presence of CVA7 or CBF9 colorectal cancer-associated proteins. However, other interstitial fluids, as well as cerebrospinal fluid also provide good samples in which to detect CVA7 or CBF9 proteins. Non-circulating fluids may also provide samples in which CVA7 and/or CBF9 proteins can be detected. Examples of non-circulating fluids include, but are not limited to fluids such as urine and sputum.

In another embodiment CVA7 and CBF9 can be measured in biopsy samples using known histological methods.

In one aspect, the expression levels of CVA7 and CBF9 gene expression are determined for different health states with respect to the colorectal cancer phenotype. Specifically, the expression levels of CVA7 and CBF9 genes in healthy individuals and in individuals with colorectal cancer are evaluated to provide understanding of the expression of CVA7 and CBF9 in colorectal cancer. There is no detectable expression of CVA7 or CBF 9 in normal colon tissues, and there is a high level expression of CVA7 or CBF9 in cancerous colon tissues. In some cases, varying severities of colorectal cancer as related to prognosis are also evaluated.

It is understood that when comparing the expression of CVA7 and/or CBF9 between an individual and a standard, the skilled artisan can make a prognosis as well as a diagnosis. It is further understood that the levels of expression of CVA7 and/or CBF9 genes which indicate the diagnosis may differ from those which indicate the prognosis.

In one embodiment, the colorectal cancer-associated proteins, antibodies, nucleic acids, modified proteins and cells containing colorectal cancer-associated sequences are used in prognosis assays. As above, expression of CVA7 and CBF9 may be correlated to colorectal cancer severity, in terms of long-term prognosis. Again, this may be done on either a protein or gene level, with the use of proteins being preferred.

Antibodies can be used to detect the colorectal cancer-associated CVA7 and CBF9 proteins by any of the previously described immunoassay techniques including ELISA, immunoblotting (Western blotting), immunoprecipitation, BIACORE technology and the like, as will be appreciated by one of ordinary skill in the art.

In another embodiment, binding assays are done. In general, purified or isolated gene product is used; that is, the gene products of CVA7 and/or CBF9 nucleic acids are made. In general, this is done as is known in the art. For example, antibodies are generated to the protein gene products, and standard immunoassays are run to determine the amount of protein present.

Positive controls and negative controls may be used in the assays. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

Once the assay is run, the data is analyzed to determine the expression levels, and changes in expression levels between healthy individuals and those individuals with colorectal cancer, or between individuals with different severities of colorectal cancer disease are compared.

As will be appreciated by those in the art, nucleic acid and protein binding agents can be attached or immobilized to a solid support. This can be accomplished in a wide variety of ways. By “immobilized” and grammatical equivalents herein is meant the association or binding between the nucleic acid probe, antibody, or other binding agent and the solid support is sufficient to be stable under the conditions of binding, washing, analysis, and removal as outlined below. The binding between the binding agent and the support can be covalent or non-covalent. By “non-covalent binding” and grammatical equivalents herein is meant one or more of electrostatic, hydrophilic, and hydrophobic interactions. Included in non-covalent binding is the covalent attachment of a molecule, such as, streptavidin to the support and the non-covalent binding of the biotinylated binding agent to the streptavidin. By “covalent binding” and grammatical equivalents herein is meant that the two moieties, the solid support and the binding agent, are attached by at least one bond, including sigma bonds, pi bonds and coordination bonds. Covalent bonds can be formed directly between the binding agent and the solid support or can be formed by a cross linker or by inclusion of a specific reactive group on either the solid support or the binding agent or both molecules. Immobilization may also involve a combination of covalent and non-covalent interactions.

In one embodiment, the oligonucleotides are synthesized as is known in the art, and then attached to the surface of the solid support. As will be appreciated by those skilled in the art, either the 5′ or 3′ terminus may be attached to the solid support, or attachment may be via an internal nucleoside. A nucleic acid probe that is functional as a binding agent in the present invention is generally single stranded but can be partially single and partially double stranded. The strandedness of the probe is dictated by the structure, composition, and properties of the target sequence. In general, the nucleic acid probes range from about 8 to about 100 bases long, with from about 10 to about 80 bases being preferred, and from about 30 to about 50 bases being particularly preferred. That is, generally whole genes are not used. In some embodiments, much longer nucleic acids can be used, up to hundreds of bases.

In one embodiment, the binding agent immobilized to a solid support is an antibody. In this case antibodies may be derivatized with bifunctional agents for the purpose of crosslinking antibodies to CVA7 and CBF9 colorectal cancer-associated sequences to a water-insoluble support matrix or surface for use in the method for identifying CVA7 and/or CBF9 proteins in serum or blood samples. Commonly used crosslinking agents include, e.g., 1,1-bis(diazo-acetyl)-2-phenylethane, glutaraldehyde, N-hydroxy-succinimide esters, for example, esters with 4-azido-salicylic acid, homobifunctional imidoesters, including disuccinimidyl esters such as 3,3′-dithiobis-(succinimidyl-propionate), bifunctional maleimides such as bis-N-maleimido-1,8-octane and agents such as methyl-3-[(p-azidophenyl)-dithio]pro-pioimi-date.

Kits for Use in Diagnostic and/or Prognostic Applications

For use in diagnostic, research, and therapeutic applications suggested above, kits are also provided by the invention. In the diagnostic and research applications such kits may include any or all of the following: assay reagents, buffers, colorectal cancer-specific nucleic acids or antibodies, hybridization probes and/or primers, antisense polynucleotides, ribozymes, dominant negative ovarian cancer polypeptides or polynucleotides, small molecules inhibitors of colorectal cancer-associated sequences etc. A therapeutic product may include sterile saline or another pharmaceutically acceptable emulsion and suspension base.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The present invention also provides for kits for screening for modulators of colorectal cancer-associated sequences. Such kits can be prepared from readily available materials and reagents. For example, such kits can comprise one or more of the following materials: a colorectal cancer-associated polypeptide or polynucleotide, reaction tubes, and instructions for testing colorectal cancer-associated activity. Optionally, the kit contains biologically active colorectal cancer protein. A wide variety of kits and components can be prepared according to the present invention, depending upon the intended user of the kit and the particular needs of the user. Diagnosis world typically involve evaluation of a plurality of genes or products. The genes will be selected based on correlations with important parameters in disease.

### EXAMPLES

### Example 1

Tissue Preparation, Labeling Chips, and Fingerprints Purifying Total RNA from Tissue Sample Using TRIzol Reagent

The tissue sample weight is first estimated. The tissue samples are homogenized in 1 ml of TRIzol per 50 mg of tissue using a homogenizer (e.g., Polytron 3100). The size of the generator/probe used depends upon the sample amount. A generator that is too large for the amount of tissue to be homogenized will cause a loss of sample and lower RNA yield. A larger generator (e.g., 20 mm) is suitable for tissue samples weighing more than 0.6 g. Fill tubes should not be overfilled. If the working volume is greater than 2 ml and no greater than 10 ml, a 15 ml polypropylene tube (Falcon 2059) is suitable for homogenization.

Tissues should be kept frozen until homogenized. The TRIzol is added directly to the frozen tissue before homogenizailon. Following homogenization, the insoluble material is removed from the homogenate by centrifugation at 7500×g for 15 min. in a Sorvall superspeed or 12,000×g for 10 min. in an Eppendorf centrifuge at 4° C. The cleared homogenate is then transferred to a new tube(s). Samples may be frozen and stored at −60 to −70° C. for at least one month or else continue with the purification.

The next process is phase separation. The homogenized samples are incubated for 5 minutes at room temperature. Then, 0.2 ml of chloroform per 1 ml of TRIzol reagent is added to the homogenization mixture. The tubes are securely capped and shaken vigorously by hand (do not vortex) for 15 seconds. The samples are then incubated at room temp. for 2-3 minutes and next centrifuged at 6500 rpm in a Sorvall superspeed for 30 min. at 4° C.

The next process is RNA Precipitation. The aqueous phase is transferred to a fresh tube. The organic phase can be saved if isolation of DNA or protein is desired. Then 0.5 ml of isopropyl alcohol is added per lml of TRIzol reagent used in the original homogenization. Then, the tubes are securely capped and inverted to mix. The samples are then incubated at room temp. for 10 minutes an centrifuged at 6500 rpm in Sorvall for 20 min. at 4° C.

The RNA is then washed. The supernatant is poured off and the pellet washed with cold 75% ethanol. 1 ml of 75% ethanol is used per 1 ml of the TRIzol reagent used in the initial homogenization. The tubes are capped securely and inverted several times to loosen pellet without vortexing. They are next centrifuged at<8000 rpm (<7500×g) for 5 minutes at 4° C.

The RNA wash is decanted. The pellet is carefully transferred to an Eppendorf tube (sliding down the tube into the new tube by use of a pipet tip to help guide it in if necessary). Tube(s) sizes for precipitating the RNA depending on the working volumes. Larger tubes may take too long to dry. Dry pellet. The RNA is then resuspended in an appropriate volume (e.g., 2-5 ug/ul) of DEPC H20. The absorbance is then measured.

The poly A+mRNA may next be purified from total RNA by other methods such as Qiagen's RNEASY® (chromatographic materials for separation of nucleic acids) kit. The poly A+mRNA is purified from total RNA by adding the OLIGOTEX® (chemicals for the purification of nucleic acids) suspension which has been heated to 37° C. and mixing prior to adding to RNA. The Elution Buffer is incubated at 70° C. If there is precipitate in the buffer, warm up the 2×Binding Buffer at 65° C. The total RNA is mixed with DEPC-treated water, 2×Binding Buffer, and OLIGOTEX® (chemicals for the purification of nucleic acids) according to Table 2 on page 16 of the OLIGOTEX® Handbook and next incubated for 3 minutes at 65° C. and 10 minutes at room temperature.

The preparation is centrifuged for 2 minutes at 14,000 to 18,000 xg, preferably, at a “soft setting,” The supernatant is removed without disturbing Oligotex pellet. A little bit of solution can be left behind to reduce the loss of OLIGOTEX®. The supernatant is saved until satisfactory binding and elution of poly A+mRNA has been found.

Then, the preparation is gently resuspended in Wash Buffer OW2 and pipetted onto the spin column and centrifuged at full speed (soft setting if possible) for 1 minute.

Next, the spin column is transferred to a new collection tube and gently resuspended in Wash Buffer OW2 and centrifuged as described herein.

Then, the spin column is transferred to a new tube and eluted with 20 to 100 ul of preheated (70° C.) Elution Buffer. The OLIGOTEX® resin is gently resuspended by pipetting up and down. The centrifugation is repeated as above and the elution repeated with fresh elution buffer or first eluate to keep the elution volume low.

The absorbance is next read to determine the yield, using diluted Elution Buffer as the blank.

Before proceeding with cDNA synthesis, the mRNA is precipitated before proceeding with cDNA synthesis, as components leftover or in the Elution Buffer from the OLIGOTEX® purification procedure will inhibit downstream enzymatic reactions of the mRNA. 0.4 vol. of 7.5 M NH4OAc+2.5 vol. of cold 100% ethanol is added and the preparation precipitated at −20° C. 1 hour to overnight (or 20-30 min. at −70° C.), and centrifuged at 14,000-16,000×g for 30 minutes at 4° C. Next, the pellet is washed with 0.5 ml of 80% ethanol (−20° C.) and then centrifuged at 14,000-16,000×g for 5 minutes at room temperature. The 80% ethanol wash is then repeated. The last bit of ethanol from the pellet is then dried without use of a speed vacuum and the pellet is then resuspended in DEPC H₂O at 1μg/μl concentration.

Alternatively the RNA may be Purified Using Other Methods (e.g., Qiagen's RNEASY® kit).

No more than 100 μg is added to the RNEASY®(chromatographic materials for separation of nucleic acids) column. The sample volume is adjusted to 100 ul with RNase-free water. 350 ul Buffer RLT and then 250 ul ethanol (100%) are added to the sample. The preparation is then mixed by pipetting and applied to an RNEASY® mini spin column for centrifugation (15 sec at>10,000 rpm). If yield is low, reapply the flowthrough to the column and centrifuge again.

Then, transfer column to a new 2 ml collection tube and add 500 ul Buffer RPE and centrifuge for 15 sec at>10,000 rpm. The flowthrough is discarded. 500 ul Buffer RPE and is then added and the preparation is centriuged for 15 sec at>10,000 rpm. The flowthrough is discarded, and the column membrane dried by centrifuging for 2 min at maximum speed. The column is transferred to a new 1.5-ml collection tube. 30-50 ul of RNase-free water is applied directly onto column membrane. The column is then centrifuged for 1 min at >10,000 rpm and the elution step repeated.

The absorbance is then read to determine yield. If necessary, the material may be ethanol precipitated with ammonium acetate and 2.5×volume 100% ethanol.

First Strand cDNA Synthesis

The first strand can be make using Gibco's “SUPERSCRIPT® Choice System for cDNA Synthesis” kit. The starting material is 5 ug of total RNA or 1 ug of polyA+mRNA1. For total RNA, 2 ul of SUPERSCRIPT® RT is used; for polyA+mRNA, 1 ul of SUPERSCRIPT® RT is used. The final volume of first strand synthesis mix is 20 ul. The RNA should be in a volume no greater than 10 ul. The RNA is incubated with 1 ul of 100 pmol T7-T24 oligo for 10 min at 70° C. followed by addition on ice of 7 μl of: 4 μl 5×1^{st} Strand Buffer, 2 ul of 0.1M DTT, and 1 ul of 10mM dNTP mix. The preparation is then incubated at 37° C. for 2 min before addition of the SUPERSCRIPT® RT followed by incubation at 37° C. for 1 hour.

Second Strand Synthesis

For the second strand synthesis, place 1 st strand reactions on ice and add: 91 ul DEPC H₂O; 30 ul 5×2nd Strand Buffer; 3 ul 10mM dNTP mix; 1 ul 10 U/ul E.coli DNA Ligase 4 ul 10 U/ul E.coli DNA Polymerase; and 1 ul 2 U/ul RNase H. Mix and incubate 2 hours at 16° C. Add 2 ul T4 DNA Polymerase. Incubate 5 min at 16° C. Add 10 ul of 0.5M EDTA.

Cleaning up cDNA

The cDNA is purified using Phenol:Chloroform:Isoamyl Alcohol (25:24:1) and Phase-Lock gel tubes. The PLG tubes are centrifuged for 30 sec at maximum speed. The cDNA mix is then transferred to PLG tube. An equal volume of phenol:chloroform:isamyl alcohol is then added, the preparation shaken vigorously (no vortexing), and centrifuged for 5 minutes at maximum speed. The top aqueous solution is transferred to a new tube and ethanol precipitated by adding 7.5×5M NH4OAc and 2.5× volume of 100% ethanol. Next, it is centrifuged immediately at room temperature for 20 min, maximum speed. The supernatant is removed, and the pellet washed with 2× with cold 80% ethanol. As much ethanol wash as possible should be removed before air drying the pellet; and resuspending it in 3 ul RNase-free water.

In vitro Transcription (IVT) and Labeling with Biotin

In vitro Transcription (IVT) and labeling with biotin is performed as follows: Pipet 1.5 ul of cDNA into a thin-wall PCR tube. Make NTP labeling mix by combining 2 ul T7 10×ATP (75 mM) (Ambion); 2 ul T7 10×GTP (75 mM) (Ambion); 1.5 ul T7 10×CTP (75 mM) (Ambion); 1.5 ul T7 10×UTP (75 mM) (Ambion); 3.75 ul 10 mM Bio-11-UTP (Boehringer-Mannheim/Roche or Enzo); 3.75 ul 10 mM Bio-16-CTP (Enzo); 2 ul 10×T7 transcription buffer (Ambion); and 2 ul 10×T7 enzyme mix (Ambion). The final volume is 20 ul. Incubate 6 hours at 37° C. in a PCR machine. The RNA can be furthered cleaned. Clean-up follows the previous instructions for RNEASY® columns or Qiagen's RNeasy protocol handbook. The cRNA often needs to be ethanol precipitated by resuspension in a volume compatible with the fragmentation step.

Fragmentation is performed as follows. 15 ug of labeled RNA is usually fragmented. Try to minimize the fragmentation reaction volume; a 10 ul volume is recommended but 20 ul is all right. Do not go higher than 20 ul because the magnesium in the fragmentation buffer contributes to precipitation in the hybridization buffer. Fragment RNA by incubation at 94 C for 35 minutes in 1×Fragmentation buffer (5×Fragmentation buffer is 200 mM Tris-acetate, pH 8.1; 500 mM KOAc; 150 mM MgOAc). The labeled RNA transcript can be analyzed before and after fragmentation. Samples can be heated to 65° C. for 15 minutes and electrophoresed on 1% agarose/TBE gels to get an approximate idea of the transcript size range.

For hybridization, 200 ul (10 ug cRNA) of a hybridization mix is put on the chip. If multiple hybridizations are to be done (such as cycling through a 5 chip set), then it is recommended that an initial hybridization mix of 300 ul or more be made. The hybridization mix is: fragment labeled RNA (50 ng/ul final conc.); 50 pM 948-b control oligo; 1.5 pM BioB; 5 pM BioC; 25 pM BioD; 100 pM CRE; 0.1 mg/ml herring sperm DNA; 0.5 mg/ml acetylated BSA; and 300 ul with 1×MES hyb buffer.

The hybridization reaction is conducted with non-biotinylated IVT (purified by RNEASY® columns) (see example 1 for steps from tissue to IVT): The following mixture is prepared:

| | |
|---|---|
| IVT antisense RNA; 4 μg: | μl |
| Random Hexamers (1 μg/μl): | 4 μl |
| H₂O: | μl |
| | 14 μ1 |

Incubate the above 14 μl mixture at 70° C. for 10 min.; then put on ice.

The Reverse transcription procedure uses the following mixture:

| | |
|---|---|
| 0.1 M DTT: | 3 μl |
| 50X dNTP mix: | 0.6 μl |
| H₂O: | 2.4 μl |
| Cy3 or Cy5 dUTP (1 mM): | 3 μl |
| SS RT II (BRL): | 1 μl |
| | 16 μl |

The above solution is added to the hybridization reaction and incubated for 30 min., 42° C. Then, 1 μl SSII is added and incubated for another hour before being placed on ice.

The 50×dNTP mix contains 25mM of cold dATP, dCTP, and dGTP,10 mM of dTTP and is made by adding 25 μl each of 100mM dATP, dCTP, and dGTP; 10 μl of 100mM dTTP to 15 μl H₂O.

RNA degradation is performed as follows. Add 86 μl H₂O, 1.5 μl 1M NaOH/2 mM EDTA and incubate at 65° C., 10 min. For U-Con 30, 500 μl TE/sample spin at 7000 g for 10 min, save flow through for purification. For Qiagen purification, suspend u-con recovered material in 500 μl buffer PB and proceed using Qiagen protocol. For DNAse digestion, add 1 μl of 1/100 dilution of DNAse/30 μl Rx and incubate at 37° C. for 15 min. Incubate at 5 min 95° C. to denature the DNAse.

Sample Preparation

For sample preparation, add Cot-1 DNA, 10 μl; 50×dNTPs, 1 p; 20×SSC, 2.3 μl; Na pyro phosphate, 7.5 μl; 10 mg/ml Herring sperm DNA; 1 μl of 1/10 dilution to 21.8 final vol. Dry in speed vac. Resuspend in 15 μl H₂O. Add 0.38 μl 10% SDS. Heat 95° C., 2 min and slow cool at room temp. for 20 min. Put on slide and hybridize overnight at 64° C. Washing after the hybridization: 3×SSC/0.03% SDS: 2 min., 37.5 ml 20×SSC+0.75 ml 10% SDS in 250 ml H₂O; 1×SSC: 5 min., 12.5 ml 20×SSC in 250 ml H₂O; 0.2×SSC: 5 min., 2.5 ml 20×SSC in 250 ml H₂O. Dry slides and scan at appropriate PMT's and channels.

### Example 2

Expression Data on Colon Cancers and Normal Tissues.

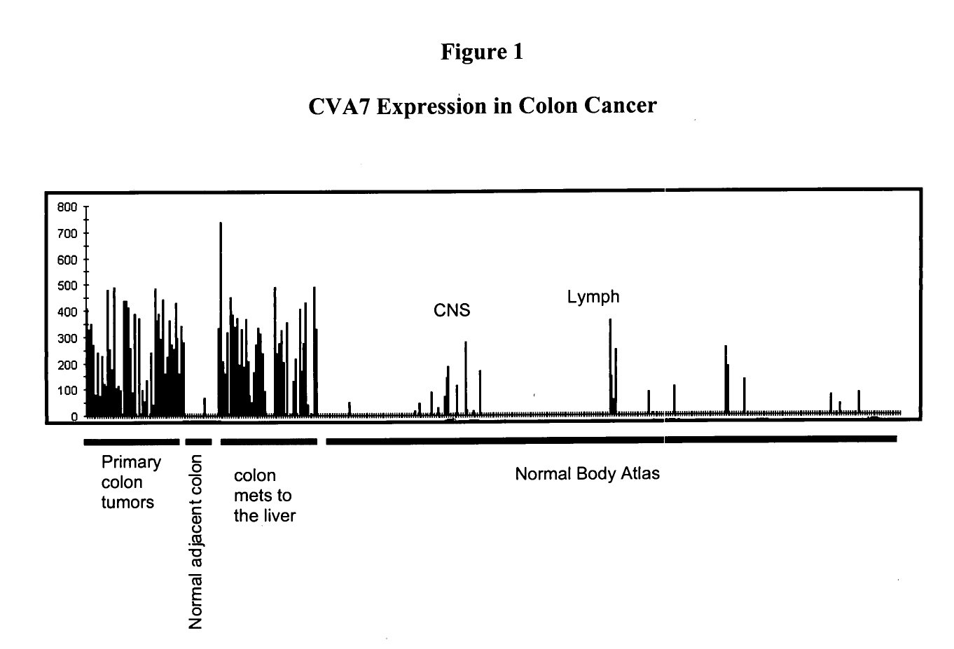

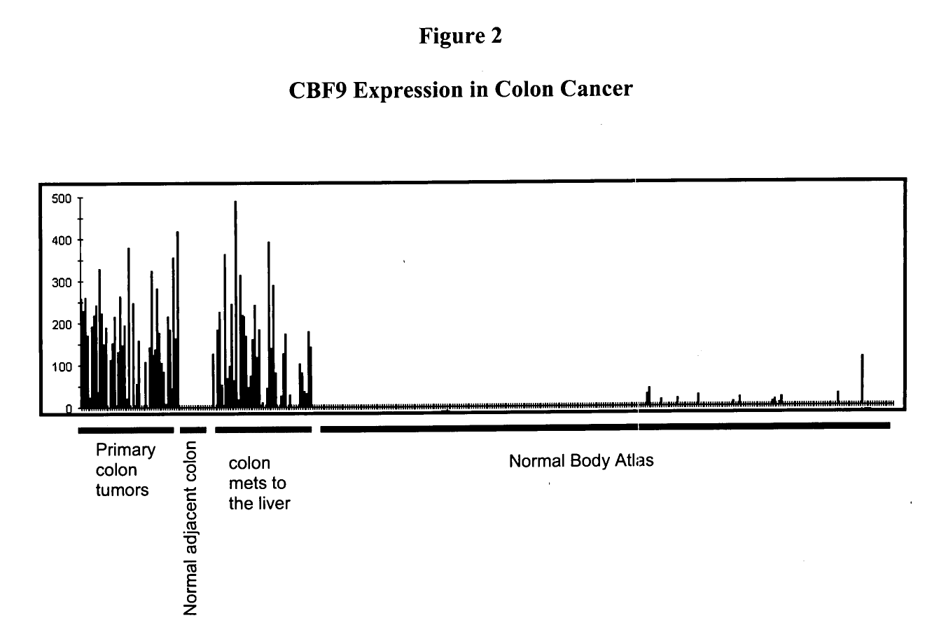

Expression studies of colon tissues and other normal tissues were performed according to Example 1. FIG. 1 shows the CVA expression in colon cancer tissues and normal body atlas. FIG. 2 shows the CBF9 expression in colon cancer tissues and normal body atlas.

### Example 3

Detection of Secreted CBF9 and CVA7

His-tagged versions of the genes for CBF9 and CVA7 were transfected into a colon cancer cell line (Vaco 364). These cell lines were then grown in tissue culture in vitro and as xenografts in severe combined immunodeficient (SCID) mice in vivo. The media from the cells grown in vitro and mouse serum from animals bearing xenograft tumors were then analyzed for the presence of secreted protein. To detect secreted protein, an antibody that binds to the His-tag on the recombinant proteins was used. Our results show that both CVA7 and CBF9 were secreted into the media by transfected cells grown in culture, but not in control cells that did not express the target genes. Similarly, both proteins were detected in the serum of mice carrying tumors of transfected cells, but not in the serum of control mice.

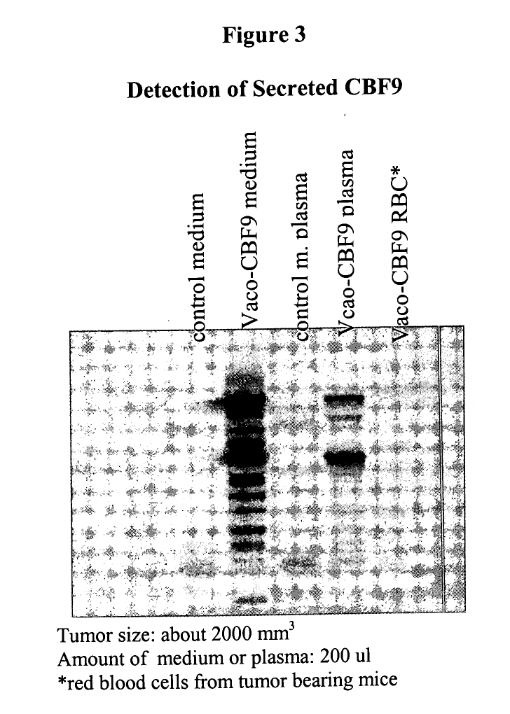

FIG. 3 shows the detection of secreted CBF9 in Vaco-CBF9 medium, Vaco-CBF9 plasma, and Vaco-CBF9 RBC, but not in control medium, or control medium plasma.

### Example 3

Analysis of CVA7 and CBF9 Expression in Blood Using Antibody-sandwich ELISA to Detect the Soluble Antigens

Blood samples are obtained from a patient using methods outlined in U.S. Pat. No. 6,283,926, the content of which is herein incorporated by reference.

Molecular profiles of various serum and blood samples are determined by performance of antibody-sandwich ELISA to detect the soluble antigens. Methods for conducting antibody-sandwich ELISA can be found in: Current Protocols in Molecular Biology (1998) Vol. 2, page 11.2.8 F.M. Ausubel et al. eds.

Detection of CVA7 and/or CBF9 protien are diagnostic of colorectal cancer.

It is understood that the examples described above in no way serve to limit the true scope of this invention, but rather are presented for illustrative purposes. All publications, sequences of accession numbers, and patent applications cited in this specification are herein incorporated by reference as if each individual publication or patent application were specifically and individually indicated to be incorporated by reference.

TABLE 2

CBF9 and CVA7 DNA and Protein Sequences
Table 2 shows the nucleotide and protein sequences for CBF9
and CVA7 genes. The CVA7 sequences shown here comprise two
sequence variants of the gene.

CBF9 DNA sequence (SEQ ID NO: 1)

| | |
|---|---|
| Unigene number: | Hs.157601 |
| Probeset Accession #: | W07459 |
| Nucleic Acid Accession #: | AC005383 |
| Coding Sequence: | 328-2751 (underlined sequences correspond |
| | to start and stop codons) |

1 11 21 31 41 51
| | | | | |

| | |
|---|---|
| GACAGTGTTC GCGGCTGCAC CGCTCGGAGG CTGGGTGACC CGCGTAGAAG TGAAGTACTT | 60 |
| TTTTATTTGC AGACCTGGGC CGATGCCGCT TTAAAAAACG CGAGGGGCTC TATGCACCTC | 120 |
| CCTGGCGGTA GTTCCTCCGA CCTCAGCCGG GTCGGGTCGT GCCGCCCTCT CCCAGGAGAG | 180 |
| ACAAACAGGT GTCCCACGTG GCAGCCGCGC CCCGGGCGCC CCTCCTGTGA TCCCGTAGCG | 240 |
| CCCCCTGGCC CGAGCCGCGC CCGGGTCTGT GAGTAGAGCC GCCCGGGCAC CGAGCGCTGG | 300 |
| TCGCCGCTCT CCTTCCGTTA TATCAACATG CCCCCTTTCC TGTTGCTGGA GGCCGTCTGT | 360 |
| GTTTTCCTGT TTTCCAGAGT GCCCCCATCT CTCCCTCTCC AGGAAGTCCA TGTAAGCAAA | 420 |
| GAAACCATCG GGAAGATTTC AGCTGCCAGC AAAATGATGT GGTGCTCGGC TGCAGTGGAC | 480 |
| ATCATGTTTC TGTTAGATGG GTCTAACAGC GTCGGGAAAG GGAGCTTTGA AAGGTCCAAG | 540 |
| CACTTTGCCA TCACAGTCTG TGACGGTCTG GACATCAGCC CCGAGAGGGT CAGAGTGGGA | 600 |
| GCATTCCAGT TCAGTTCCAC TCCTCATCTG GAATTCCCCT TGGATTCATT TTCAACCCAA | 660 |
| CAGGAAGTGA AGGCAAGAAT CAAGAGGATG GTTTTCAAAG GAGGGCGCAC GGAGACGGAA | 720 |
| CTTGCTCTGA AATACCTTCT GCACAGAGGG TTGCCTGGAG GCAGAAATGC TTCTGTGCCC | 780 |
| CAGATCCTCA TCATCGTCAC TGATGGGAAG TCCCAGGGGG ATGTGGCACT GCCATCCAAG | 840 |
| CAGCTGAAGG AAAGGGGTGT CACTGTGTTT GCTGTGGGGG TCAGGTTTCC CAGGTGGGAG | 900 |
| GAGCTGCATG CACTGGCCAG CGAGCCTAGA GGGCAGCACG TGCTGTTGGC TGAGCAGGTG | 960 |
| GAGGATGCCA CCAACGGCCT CTTCAGCACC CTCAGCAGCT CGGCCATCTG CTCCAGCGCC | 1020 |
| ACGCCAGACT GCAGGGTCGA GGCTCACCCC TGTGAGCACA GGACGCTGGA GATGGTCCGG | 1080 |
| GAGTTCGCTG GCAATGCCCC ATGCTGGAGA GGATCGCGGC GGACCCTTGC GGTGCTGGCT | 1140 |
| GCACACTGTC CCTTCTACAG CTGGAAGAGA GTGTTCCTAA CCCACCCTGC CACCTGCTAC | 1200 |
| AGGACCACCT GCCCAGGCCC CTGTGACTCG CAGCCCTGCC AGAATGGAGG CACATGTGTT | 1260 |
| CCAGAAGGAC TGGACGGCTA CCAGTGCCTC TGCCCGCTGG CCTTTGGAGG GGAGGCTAAC | 1320 |
| TGTGCCCTGA AGCTGAGCCT GGAATGCAGG GTCGACCTCC TCTTCCTGCT GGACAGCTCT | 1380 |
| GCGGGCACCA CTCTGGACGG CTTCCTGCGG GCCAAAGTCT TCGTGAAGCG GTTTGTGCGG | 1440 |
| GCCGTGCTGA GCGAGGACTC TCGGGCCCGA GTGGGTGTGG CCACATACAG CAGGGAGCTG | 1500 |
| CTGGTGGCGG TGCCTGTGGG GGAGTACCAG GATGTGCCTG ACCTGGTCTG GAGCCTCGAT | 1560 |
| GGCATTCCCT TCCGTGGTGG CCCCACCCTG ACGGGCAGTG CCTTGCGGCA GGCGGCAGAG | 1620 |
| CGTGGCTTCG GGAGCGCCAC CAGGACAGGC CAGGACCGGC CACGTAGAGT GGTGGTTTTG | 1680 |
| CTCACTGAGT CACACTCCGA GGATGAGGTT GCGGGCCCAG CGCGTCACGC AAGGGCGCGA | 1740 |
| GAGCTGCTCC TGCTGGGTGT AGGCAGTGAG GCCGTGCGGG CAGAGCTGGA GGAGATCACA | 1800 |
| GGCAGCCCAA AGCATGTGAT GGTCTACTCG GATCCTCAGG ATCTGTTCAA CCAAATCCCT | 1860 |
| GAGCTGCAGG GGAAGCTGTG CAGCCGGCAG CGGCCAGGGT GCCGGACACA AGCCCTGGAC | 1920 |
| CTCGTCTTCA TGTTGGACAC CTCTGCCTCA GTAGGGCCCG AGAATTTTGC TCAGATGCAG | 1980 |
| AGCTTTGTGA GAAGCTGTGC CCTCCAGTTT GAGGTGAACC CTGACGTGAC ACAGGTCGGC | 2040 |
| CTGGTGGTGT ATGGCAGCCA GGTGCAGACT GCCTTCGGGC TGGACACCAA ACCCACCCGG | 2100 |
| GCTGCGATGC TGCGGGCCAT TAGCCAGGCC CCCTACCTAG GTGGGGTGGG CTCAGCCGGC | 2160 |
| ACCGCCCTGC TGCACATCTA TGACAAAGTG ATGACCGTCC AGAGGGGTGC CCGGCCTGGT | 2220 |
| GTCCCCAAAG CTGTGGTGGT GCTCACAGGC GGGAGAGGCG CAGAGGATGC AGCCGTTCCT | 2280 |
| GCCCAGAAGC TGAGGAACAA TGGCATCTCT GTCTTGGTCG TGGGCGTGGG GCCTGTCCTA | 2340 |
| AGTGAGGGTC TGCGGAGGCT TGCAGGTCCC CGGGATTCCC TGATCCACGT GGCAGCTTAC | 2400 |
| GCCGACCTGC GGTACCACCA GGACGTGCTC ATTGAGTGGC TGTGTGGAGA AGCCAAGCAG | 2460 |
| CCAGTCAACC TCTGCAAACC CAGCCCGTGC ATGAATGAGG GCAGCTGCGT CCTGCAGAAT | 2520 |
| GGGAGCTACC GCTGCAAGTG TCGGGATGGC TGGGAGGGCC CCCACTGCGA GAACCGTGAG | 2580 |
| TGGAGCTCTT GCTCTGTATG TGTGAGCCAG GGATGGATTC TTGAGACGCC CCTGAGGCAC | 2640 |
| ATGGCTCCCG TGCAGGAGGG CAGCAGCCGT ACCCCTCCCA GCAACTACAG AGAAGGCCTG | 2700 |
| GGCACTGAAA TGGTGCCTAC CTTCTGGAAT GTCTGTGCCC CAGGTCCTTA GAATGTCTGC | 2760 |
| TTCCCGCCGT GGCCAGGACC ACTATTCTCA CTGAGGGAGG AGGATGTCCC AACTGCAGCC | 2820 |
| ATGCTGCTTA GAGACAAGAA AGCAGCTGAT GTCACCCACA AACGATGTTG TTGAAAAGTT | 2880 |
| TTGATGTGTA AGTAAATACC CACTTTCTGT ACCTGCTGTG CCTTGTTGAG GCTATGTCAT | 2940 |
| CTGCCACCTT TCCCTTGAGG ATAAACAAGG GGTCCTGAAG ACTTAAATTT AGCGGCCTGA | 3000 |
| CGTTCCTTTG CACACAATCA ATGCTCGCCA GAATGTTGTT GACACAGTAA TGCCCAGCAG | 3060 |
| AGGCCTTTAC TAGAGCATCC TTTGGACGGC GAAGGCCACG GCCTTTCAAG ATGGAAAGCA | 3120 |
| GCAGCTTTTC CACTTCCCCA GAGACATTCT GGATGCATTT GCATTGAGTC TGAAAGGGGG | 3180 |
| CTTGAGGGAC GTTTGTGACT TCTTGGCGAC TGCCTTTTGT GTGTGGAAGA GACTTGGAAA | 3240 |
| GGTCTCAGAC TGAATGTGAC CAATTAACCA GCTTGGTTGA TGATGGGGGA GGGGCTGAGT | 3300 |
| TGTGCATGGG CCCAGGTCTG GAGGGCCACG TAAAATCGTT CTGAGTCGTG AGCAGTGTCC | 3360 |
| ACCTTGAAGG TCTTC | |

CBF9 Protein sequence (SEQ ID NO: 2)

| | |
|---|---|
| Gene name: | ESTs |
| Unigene number: | Hs.157601 |
| Protein Accession #: | none found |
| Signal sequence: | 1-17 |
| Transmembrane domains: | none found |
| VGW domains: | 49-223; 341-518; 529-706 |
| EGF domains: | 298-333; 715-748 |
| Cellular Localization: | plasma membrane |

1 11 21 31 41 51
| | | | | |

| | |
|---|---|
| MPPFLLLEAV CVFLFSRVPP SLPLQEVHVS KETIGKISAA SKMMWCSAAV DIMFLLDGSN | 60 |
| SVGKGSFERS KHFAITVCDG LDISPERVRV GAFQFSSTPH LEFPLDSFST QQEVKARIKR | 120 |
| MVFKGGRTET ELALKYLLHR GLPGGRNASV PQILIIVTDG KSQGDVALPS KQLKERGVTV | 180 |
| FAVGVRFPRW EELHALASEP RGQHVLLAEQ VEDATNGLFS TLSSSAICSS ATPDCRVEAH | 240 |
| PCEHRTLEMV REFAGNAPCW RGSRRTLAVL AAHCPFYSWK RVFLTHPATC YRTTCPGPCD | 300 |
| SQPCQNGGTC VPEGLDGYQC LCPLAFGGEA NCALKLSLEC RVDLLFLLDS SAGTTLDGFL | 360 |
| RAKVFVKRFV RAVLSEDSRA RVGVATYSRE LLVAVPVGEY QDVPDLVWSL DGIPFRGGPT | 420 |
| LTGSALRQAA ERGFGSATRT GQDRPRRVVV LLTESHSEDE VAGPARHARA RELLLLGVGS | 480 |
| EAVRAELEEI TGSPKHVMVY SDPQDLFNQI PELQGKLCSR QRPGCRTQAL DLVFMLDTSA | 540 |
| SVGPENFAQM QSFVRSCALQ FEVNPDVTQV GLVVYGSQVQ TAFGLDTKPT RAAMLRAISQ | 600 |
| APYLGGVGSA GTALLHIYDK VMTVQRGARP GVPKAVVVLT GGRGAEDAAV PAQKLRNNGI | 660 |
| SVLVVGVGPV LSEGLRRLAG PRDSLIHVAA YADLRYHQDV LIEWLCGEAK QPVNLCKPSP | 720 |
| CMNEGSCVLQ NGSYRCKCRD GWEGPHCENR EWSSCSVCVS QGWILETPLR HMAPVQEGSS | 780 |
| RTPPSNYREG LGTEMVPTFW NVCAPGP | |

CVA7 DNA and Protein Sequences
CVA7 DNA sequence (SEQ ID NO: 3)

| | |
|---|---|
| Nucleic Acid Accession #: | XM_051860.2 |
| Coding sequence: | 52..3042 |

1 11 21 31 41 51
| | | | | |

| | |
|---|---|
| GCTCACCCAG GAAAAATATG CAATCGTCCC ATTGATATAC AGGCCACTAC AATGGATGGA | 60 |
| GTTAACCTCA GCACCGAGGT TGTCTACAAA AAAGGCCAGG ATTATAGGTT TGCTTGCTAC | 120 |
| GACCGGGGCA GAGCCTGCCG GAGCTACCGT GTACGGTTCC TCTGTGGGAA GCCTGTGAGG | 180 |
| CCCAAACTCA CAGTCACCAT TGACACCAAT GTGAACAGCA CCATTCTGAA CTTGGAGGAT | 240 |
| AATGTACAGT CATGGAAACC TGGAGATACC CTGGTCATTG CCAGTACTGA TTACTCCATG | 300 |
| TACCAGGCAG AAGAGTTCCA GGTGCTTCCC TGCAGATCCT GCGCCCCCAA CCAGGTCAAA | 360 |
| GTGGCAGGGA AACCAATGTA CCTGCACATC GGGGAGGAGA TAGACGGCGT GGACATGCGG | 420 |
| GCGGAGGTTG GGCTTCTGAG CCGGAACATC ATAGTGATGG GGGAGATGGA GGACAAATGC | 480 |
| TACCCCTACA GAAACCACAT CTGCAATTTC TTTGACTTCG ATACCTTTGG GGGCCACATC | 540 |
| AAGTTTGCTC TGGGATTTAA GGCAGCACAC TTGGAGGGCA CGGAGCTGAA GCATATGGGA | 600 |
| CAGCAGCTGG TGGGTCAGTA CCCGATTCAC TTCCACCTGG CCGGTGATGT AGACGAAAGG | 660 |
| GGAGGTTATG ACCCACCCAC ATACATCAGG GACCTCTCCA TCCATCATAC ATTCTCTCGC | 720 |
| TGCGTCACAG TCCATGGCTC CAATGGCTTG TTGATCAAGG ACGTTGTGGG CTATAACTCT | 780 |
| TTGGGCCACT GCTTCTTCAC GGAAGATGGG CCGGAGGAAC GCAACACTTT TGACCACTGT | 840 |
| CTTGGCCTCC TTGTCAAGTC TGGAACCCTC CTCCCCTCGG ACCGTGACAG CAAGATGTGC | 900 |
| AAGATGATCA CAGGAGACTC CTACCCAGGG TACATCCCCA AGCCCAGGCA AGACTGCAAT | 960 |
| GCTGTGTCCA CCTTCTGGAT GGCCAATCCC AACAACAACC TCATCAACTG TGCCGCTGCA | 1020 |
| GGATCTGAGG AAACTGGATT TTGGTTTATT TTTCACCACG TACCAACGGG CCCCTCCGTG | 1080 |
| GGAATGTACT CCCCAGGTTA TTCAGAGCAC ATTCCACTGG GAAAATTCTA TAACAACCGA | 1140 |
| GCACATTCCA ACTACCGGGC TGGCATGATC ATAGACAACG GAGTCAAAAC CACCGAGGCC | 1200 |
| TCTGCCAAGG ACAAGCGGCC GTTCCTCTCA ATCATCTCTG CCAGATACAG CCCTCACCAG | 1260 |
| GACGCCGACC CGCTGAAGCC CCGGGAGCCG GCCATCATCA GACACTTCAT TGCCTACAAG | 1320 |
| AACCAGGACC ACGGGGCCTG GCTGCGCGGC GGGGATGTGT GGCTGGACAG CTGCCGGTTT | 1380 |
| GCTGACAATG GCATTGGCCT GACCCTGGCC AGTGGTGGAA CCTTCCCGTA TGACGACGGC | 1440 |
| TCCAAGCAAG AGATAAAGAA CAGCTTGTTT GTTGGCGAGA GTGGCAACGT GGGGACGGAA | 1500 |
| ATGATGGACA ATAGGATCTG GGGCCCTGGC GGCTTGGACC ATAGCGGAAG GACCCTCCCT | 1560 |
| ATAGGCCAGA ATTTTCCAAT TAGAGGAATT CAGTTATATG ATGGCCCCAT CAACATCCAA | 1620 |
| AACTGCACTT TCCGAAAGTT TGTGGCCCTG GAGGGCCGGC ACACCAGCGC CCTGGCCTTC | 1680 |
| CGCCTGAATA ATGCCTGGCA GAGCTGCCCC CATAACAACG TGACCGGCAT TGCCTTTGAG | 1740 |
| GACGTTCCGA TTACTTCCAG AGTGTTCTTC GGAGAGCCTG GGCCCTGGTT CAACCAGCTG | 1800 |
| GACATGGATG GGGATAAGAC ATCTGTGTTC CATGACGTCG ACGGCTCCGT GTCCGAGTAC | 1860 |
| CCTGGCTCCT ACCTCACGAA GAATGACAAC TGGCTGGTCC GGCACCCAGA CTGCATCAAT | 1920 |
| GTTCCCGACT GGAGAGGGGC CATTTGCAGT GGGTGCTATG CACAGATGTA CATTCAAGCC | 1980 |
| TACAAGACCA GTAACCTGCG AATGAAGATC ATCAAGAATG ACTTCCCCAG CCACCCTCTT | 2040 |
| TACCTGGAGG GGGCGCTCAC CAGGAGCACC CATTACCAGC AATACCAACC GGTTGTCACC | 2100 |
| CTGCAGAAGG GCTACACCAT CCACTGGGAC CAGACGGCCC CCGCCGAACT CGCCATCTGG | 2160 |
| CTCATCAACT TCAACAAGGG CGACTGGATC CGAGTGGGGC TCTGCTACCC GCGAGGCACC | 2220 |
| ACATTCTCCA TCCTCTCGGA TGTTCACAAT CGCCTGCTGA AGCAAACGTC CAAGACGGGC | 2280 |
| GTCTTCGTGA GGACCTTGCA GATGGACAAA GTGGAGCAGA GCTACCCTGG CAGGAGCCAC | 2340 |
| TACTACTGGG ACGAGGACTC AGGGCTGTTG TTCCTGAAGC TGAAAGCTCA GAACGAGAGA | 2400 |
| GAGAAGTTTG CTTTCTGCTC CATGAAAGGC TGTGAGAGGA TAAAGATTAA AGCTCTGATT | 2460 |
| CCAAAGAACG CAGGCGTCAG TGACTGCACA GCCACAGCTT ACCCCAAGTT CACCGAGAGG | 2520 |
| GCTGTCGTAG ACGTGCCGAT GCCCAAGAAG CTCTTTGGTT CTCAGCTGAA AACAAAGGAC | 2580 |
| CATTTCTTGG AGGTGAAGAT GGAGAGTTCC AAGCAGCACT TCTTCCACCT CTGGAACGAC | 2640 |
| TTCGCTTACA TTGAAGTGGA TGGGAAGAAG TACCCCAGTT CGGAGGATGG CATCCAGGTG | 2700 |
| GTGGTGATTG ACGGGAACCA AGGGCGCGTG GTGAGCCACA CGAGCTTCAG GAACTCCATT | 2760 |
| CTGCAAGGCA TACCATGGCA GCTTTTCAAC TATGTGGCGA CCATCCCTGA CAATTCCATA | 2820 |
| GTGCTTATGG CATCAAAGGG AAGATACGTC TCCAGAGGCC CATGGACCAG AGTGCTGGAA | 2880 |
| AAGCTTGGGG CAGACAGGGG TCTCAAGTTG AAAGAGCAAA TGGCATTCGT TGGCTTCAAA | 2940 |
| GGCAGCTTCC GGCCCATCTG GGTGACACTG GACACTGAGG ATCACAAAGC CAAAATCTTC | 3000 |
| CAAGTTGTGC CCATCCCTGT GGTGAAGAAG AAGAAGTTGT GAGGACAGCT GCCGCCCGGT | 3060 |
| GCCACCTCGT GGTAGACTAT GACGGTGACT CTTGGCAGCA GACCAGTGGG GGATGGCTGG | 3120 |
| GTCCCCCAGC CCCTGCCAGC AGCTGCCTGG GAAGGCCGTG TTTCAGCCCT GATGGGCCAA | 3180 |
| GGGAAGGCTA TCAGAGACCC TGGTGCTGCC ACCTGCCCCT ACTCAAGTGT CTACCTGGAG | 3240 |
| CCCCTGGGGC GGTGCTGGCC AATGCTGGAA ACATTCACTT TCCTGCAGCC TCTTGGGTGC | 3300 |
| TTCTCTCCTA TCTGTGCCTC TTCAGTGGGG GTTTGGGGAC CATATCAGGA GACCTGGGTT | 3360 |
| GTGCTGACAG CAAAGATCCA CTTTGGCAGG AGCCCTGACC CAGCTAGGAG GTAGTCTGGA | 3420 |
| GGGCTGGTCA TTCACAGATC CCCATGGTCT TCAGCAGACA AGTGAGGGTG GTAAATGTAG | 3480 |
| GAGAAAGAGC CTTGGCCTTA AGGAAATCTT TACTCCTGTA AGCAAGAGCC AACCTCACAG | 3540 |
| GATTAGGAGC TGGGGTAGAA CTGGCTATCC TTGGGGAAGA GGCAAGCCCT GCCTCTGGCC | 3600 |
| GTGTCCACCT TTCAGGAGAC TTTGAGTGGC AGGTTTGGAC TTGGACTAGA TGACTCTCAA | 3660 |
| AGGCCCTTTT AGTTCTGAGA TTCCAGAAAT CTGCTGCATT TCACATGGTA CCTGGAACCC | 3720 |
| AACAGTTCAT GGATATCCAC TGATATCCAT GATGCTGGGT GCCCCAGCGC ACACGGGATG | 3780 |
| GAGAGGTGAG AACTAATGCC TAGCTTGAGG GGTCTGCAGT CCAGTAGGGC AGGCAGTCAG | 3840 |
| GTCCATGTGC ACTGCAATGC CAGGTGGAGA AATCACAGAG AGGTAAAATG GAGGCCAGTG | 3900 |
| CCATTTCAGA GGGGAGGCTC AGGAAGGCTT CTTGCTTACA GGAATGAAGG CTGGGGGCAT | 3960 |
| TTTGCTGGGG GGAGATGAGG CAGCCTCTGG AATGGCTCAG GGATTCAGCC CTCCCTGCCG | 4020 |
| CTGCCTGCTG AAGCTGGTGA CTACGGGGTC GCCCTTTGCT CACGTCTCTC TGGCCCACTC | 4080 |
| ATGATGGAGA AGTGTGGTCA GAGGGGAGCA ATGGGCTTTG CTGCTTATGA GCACAGAGGA | 4140 |
| ATTCAGTCCC CAGGCAGCCC TGCCTCTGAC TCCAAGAGGG TGAAGTCCAC AGAAGTGAGC | 4200 |
| TCCTGCCTTA GGGCCTCATT TGCTCTTCAT CCAGGGAACT GAGCACAGGG GGCCTCCAGG | 4260 |
| AGACCCTAGA TGTGCTCGTA CTCCCTCGGC CTGGGATTTC AGAGCTGGAA ATATAGAAAA | 4320 |
| TATCTAGCCC AAAGCCTTCA TTTTAACAGA TGGGGAAAGT GAGCCCCCAA GATGGGAAAG | 4380 |
| AACCACACAG CTAAGGGAGG GCCTGGGGAG CCCCACCCTA GCCCTTGCTG CCACACCACA | 4440 |
| TTGCCTCAAC AACCGGCCCC AGAGTGCCCA GGCACTCCTG AGGTAGCTTC TGGAAATGGG | 4500 |
| GACAAGTCCC CTCGAAGGAA AGGAAATGAC TAGAGTAGAA TGACAGCTAG CAGATCTCTT | 4560 |
| CCCTCCTGCT CCCAGCGCAC ACAAACCCGC CCTCCCCTTG GTGTTGGCGG TCCCTGTGGC | 4620 |
| CTTCACTTTG TTCACTACCT GTCAGCCCAG CCTGGGTGCA CAGTAGCTGC AACTCCCCAT | 4680 |
| TGGTGCTACC TGGCTCTCCT GTCTCTGCAG CTCTACAGGT GAGGCCCAGC AGAGGGAGTA | 4740 |
| GGGCTCGCCA TGTTTCTGGT GAGCCAATTT GGCTGATCTT GGGTGTCTGA ACAGCTATTG | 4800 |
| GGTCCACCCC AGTCCCTTTC AGCTGCTGCT TAATGCCCTG CTCTCTCCCT GGCCCACCTT | 4860 |
| ATAGAGAGCC CAAAGAGCTC CTGTAAGAGG GAGAACTCTA TCTGTGGTTT ATAATCTTGC | 4920 |
| ACGAGGCACC AGAGTCTCCC TGGGTCTTGT GATGAACTAC ATTTATCCCC TTTCCTGCCC | 4980 |
| CAACCACAAA CTCTTTCCTT CAAAGAGGGC CTGCCTGGCT CCCTCCACCC AACTGCACCC | 5040 |
| ATGAGACTCG GTCCAAGAGT CCATTCCCCA GGTGGGAGCC AACTGTCAGG GAGGTCTTTC | 5100 |
| CCACCAAACA TCTTTCAGCT GCTGGGAGGT GACCATAGGG CTCTGCTTTT AAAGATATGG | 5160 |
| CTGCTTCAAA GGCCAGAGTC ACAGGAAGGA CTTCTTCCAG GGAGATTAGT GGTGATGGAG | 5220 |
| AGGAGAGTTA AAATGACCTC ATGTCCTTCT TGTCCACGGT TTTGTTGAGT TTTCACTCTT | 5280 |
| CTAATGCAAG GGTCTCACAC TGTGAACCAC TTAGGATGTG ATCACTTTCA GGTGGCCAGG | 5340 |
| AATGTTGAAT GTCTTTGGCT CAGTTCATTT AAAAAAGATA TCTATTTGAA AGTTCTCAGA | 5400 |
| GTTGTACATA TGTTTCACAG TACAGGATCT GTACATAAAA GTTTCTTTCC TAAACCATTC | 5460 |
| ACCAAGAGCC AATATCTAGG CATTTTCTTG GTAGCACAAA TTTTCTTATT GCTTAGAAAA | 5520 |
| TTGTCCTCCT TGTTATTTCT GTTTGTAAGA CTTAAGTGAG TTAGGTCTTT AAGGAAAGCA | 5580 |
| ACGCTCCTCT GAAATGCTTG TCTTTTTTCT GTTGCCGAAA TAGCTGGTCC TTTTTCGGGA | 5640 |
| GTTAGATGTA TAGAGTGTTT GTATGTAAAC ATTTCTTGTA GGCATCACCA TGAACAAAGA | 5700 |
| TATATTTTCT ATTTATTTAT TATATGTGCA CTTCAAGAAG TCACTGTCAG AGAAATAAAG | 5760 |
| AATTGTCTTA AATGTCAAAA AAAAAAAAAA AAAAAAAAAA AAAAAAAA | |

CVA7 Protein sequence (SEQ ID NO: 4)

| | |
|---|---|
| Protein Accession #: | XP_051860.2 |

1 11 21 31 41 51
| | | | | |

| | |
|---|---|
| MDGVNLSTEV VYKKGQDYRF ACYDRGRACR SYRVRFLCGK PVRPKLTVTI DTNVNSTILN | 60 |
| LEDNVQSWKP GDTLVIASTD YSMYQAEEFQ VLPCRSCAPN QVKVAGKPMY LHIGEEIDGV | 120 |
| DMRAEVGLLS RNIIVMGEME DKCYPYRNHI CNFFDFDTFG GHIKFALGFK AAHLEGTELK | 180 |
| HMGQQLVGQY PIHFHLAGDV DERGGYDPPT YIRDLSIHHT FSRCVTVHGS NGLLIKDVVG | 240 |
| YNSLGHCFFT EDGPEERNTF DHCLGLLVKS GTLLPSDRDS KMCKMITGDS YPGYIPKPRQ | 300 |
| DCNAVSTFWM ANPNNNLINC AAAGSEETGF WFIFHHVPTG PSVGMYSPGY SEHIPLGKFY | 360 |
| NNRAHSNYRA GMIIDNGVKT TEASAKDKRP FLSIISARYS PHQDADPLKP REPAIIRHFI | 420 |
| AYKNQDHGAW LRGGDVWLDS CRFADNGIGL TLASGGTFPY DDGSKQEIKN SLFVGESGNV | 480 |
| GTEMMDNRIW GPGGLDHSGR TLPIGQNFPI RGIQLYDGPI NIQNCTFRKF VALEGRHTSA | 540 |
| LAFRLNNAWQ SCPHNNVTGI AFEDVPITSR VFFGEPGPWF NQLDMDGDKT SVFHDVDGSV | 600 |
| SEYPGSYLTK NDMWLVRHPD CINVPDWRGA ICSGCYAQMY IQAYKTSNLR MKIIKNDFPS | 660 |
| HPLYLEGALT RSTHYQQYQP VVTLQKGYTI HWDQTAPAEL AIWLINFNKG DWIRVGLCYP | 720 |
| RGTTFSILSD VHNRLLKQTS KTGVFVRTLQ MDKVEQSYPG RSHYYWDEDS GLLFLKLKAQ | 780 |
| NEREKFAFCS MKGCERIKIK ALIPKNAGVS DCTATAYPKF TERAVVDVPM PKKLFGSQLK | 840 |
| TKDHFLEVKM ESSKQHFFHL WNDFAYIEVD GKKYPSSEDG IQVVVIDGNQ GRVVSHTSFR | 900 |
| NSILQGIPWQ LFNYVATIPD NSIVLMASKG RYVSRGPWTR VLEKLGADRG LKLKEQMAFV | 960 |
| GFKGSFRPIW VTLDTEDHKA KIFQVVPIPV VKKKKL | |

CVA7 variant DNA sequence (SEQ ID NO:5)

| | |
|---|---|
| Nucleic Acid Accession #: | Eos sequence |
| Coding sequence: | 261..2861 |

1 11 21 31 41 51
| | | | | |

| | |
|---|---|
| GAGCTAGCGC TCAAGCAGAG CCCAGCGCGG TGCTATCGGA CAGAGCCTGG CGAGCGCAAG | 60 |
| CGGCGCGGGG AGCCAGCGGG GCTGAGCGCG GCCAGGGTCT GAACCCAGAT TTCCCAGACT | 120 |
| AGCTACCACT CCGCTTGCCC ACGCCCCGGG AGCTCGCGGC GCCTGGCGGT CAGCGACCAG | 180 |
| ACGTCCGGGG CCGCTGCGCT CCTGGCCCGC GAGGCGTGAC ACTGTCTCGG CTACAGACCC | 240 |
| AGAGGGAGCA CACTGCCAGG ATGGGAGCTG CTGGGAGGCA GGACTTCCTC TTCAAGGCCA | 300 |
| TGCTGACCAT CAGCTGGCTC ACTCTGACCT GCTTCCCTGG GGCCACATCC ACAGTGGCTG | 360 |
| CTGGGTGCCC TGACCAGAGC CCTGAGTTGC AACCCTGGAA CCCTGGCCAT GACCAAGACC | 420 |
| ACCATGTGCA TATCGGCCAG GGCAAGACAC TGCTGCTCAC CTCTTCTGCC ACGGTCTATT | 480 |
| CCATCCACAT CTCAGAGGGA GGCAAGCTGG TCATTAAAGA CCACGACGAG CCGATTGTTT | 540 |
| TGCGAACCCG GCACATCCTG ATTGACAACG GAGGAGAGCT GCATGCTGGG AGTGCCCTCT | 600 |
| GCCCTTTCCA GGGCAATTTC ACCATCATTT TGTATGGAAG GGCTGATGAA GGTATTCAGC | 660 |
| CGGATCCTTA CTATGGTCTG AAGTACATTG GGGTTGGTAA AGGAGGCGCT CTTGAGTTGC | 720 |
| ATGGACAGAA AAAGCTCTCC TGGACATTTC TGAACAAGAC CCTTCACCCA GGTGGCATGG | 780 |
| CAGAAGGAGG CTATTTTTTT GAAAGGAGCT GGGGCCACCG TGGAGTTATT GTTCATGTCA | 840 |
| TCGACCCCAA ATCAGGCACA GTCATCCATT CTGACCGGTT TGACACCTAT AGATCCAAGA | 900 |
| AAGAGAGTGA ACGTCTGGTC CAGTATTTGA ACGCGGTGCC CGATGGCAGG ATCCTTTCTG | 960 |
| TTGCAGTGAA TGATGAAGGT TCTCGAAATC TGGATGACAT GGCCAGGAAG GCGATGACCA | 1020 |
| AATTGGGAAG CAAACACTTC CTGCACCTTG GATTTAGACA CCCTTGGAGT TTTCTAACTG | 1080 |
| TGAAAGGAAA TCCATCATCT TCAGTGGAAG ACCATATTGA ATATCATGGA CATCGAGGCT | 1140 |
| CTGCTGCTGC CCGGGTATTC AAATTGTTCC AGACAGAGCA TGGCGAATAT TTCAATGTTT | 1200 |
| CTTTGTCCAG TGAGTGGGTT CAAGACGTGG AGTGGACGGA GTGGTTCGAT CATGATAAAG | 1260 |
| TATCTCAGAC TAAAGGTGGG GAGAAAATTT CAGACCTCTG GAAAGCTCAC CCAGGAAAAA | 1320 |
| TATGCAATCG TCCCATTGAT ATACAGGCCA CTACAATGGA TGGAGTTAAC CTCAGCACCG | 1380 |
| AGGTTGTCTA CAAAAAAGGC CAGGATTATA GGTTTGCTTG CTACGACCGG GGCAGAGCCT | 1440 |
| GCCGGAGCTA CCGTGTACGG TTCCTCTGTG GGAAGCCTGT GAGGCCCAAA CTCACAGTCA | 1500 |
| CCATTGACAC CAATGTGAAC AGCACCATTC TGAACTTGGA GGATAATGTA CAGTCATGGA | 1560 |
| AACCTGGAGA TACCCTGGTC ATTGCCAGTA CTGATTACTC CATGTACCAG GCAGAAGAGT | 1620 |
| TCCAGGTGCT TCCCTGCAGA TCCTGCGCCC CCAACCAGGT CAAAGTGGCA GGGAAACCAA | 1680 |
| TGTACCTGCA CATCGGGGAG GAGATAGACG GCGTGGACAT GCGGGCGGAG GTTGGGCTTC | 1740 |
| TGAGCCGGAA CATCATAGTG ATGGGGGAGA TGGAGGACAA ATGCTACCCC TACAGAAACC | 1800 |
| ACATCTGCAA TTTCTTTGAC TTCGATACCT TTGGGGGCCA CATCAAGTTT GCTCTGGGAT | 1860 |
| TTAAGGCAGC ACACTTGGAG GGCACGGAGC TGAAGCATAT GGGACAGCAG CTGGTGGGTC | 1920 |
| AGTACCCGAT TCACTTCCAC CTGGCCGGTG ATGTAGACGA AAGGGGAGGT TATGACCCAC | 1980 |
| CCACATACAT CAGGGACCTC TCCATCCATC ATACATTCTC TCGCTGCGTC ACAGTCCATG | 2040 |
| GCTCCAATGG CTTGTTGATC AAGGACGTTG TGGGCTATAA CTCTTTGGGC CACTGCTTCT | 2100 |
| TCACGGAAGA TGGGCCGGAG GAACGCAACA CTTTTGACCA CTGTCTTGGC CTCCTTGTCA | 2160 |
| AGTCTGGAAC CCTCCTCCCC TCGGACCGTG ACAGCAAGAT GTGCAAGATG ATCACAGAGG | 2220 |
| ACTCCTACCC AGGGTACATC CCCAAGCCCA GGCAAGACTG CAATGCTGTG TCCACCTTCT | 2280 |
| GGATGGCCAA TCCCAACAAC AACCTCATCA ACTGTGCCGC TGCAGGATCT GAGGAAACTG | 2340 |
| GATTTTGGTT TATTTTTCAC CACGTACCAA CGGGCCCCTC CGTGGGAATG TACTCCCCAG | 2400 |
| GTTATTCAGA GCACATTCCA CTGGGAAAAT TCTATAACAA CCGAGCACAT TCCAACTACC | 2460 |
| GGGCTGGCAT GATCATAGAC AACGGAGTCA AAACCACCGA GGCCTCTGCC AAGGACAAGC | 2520 |
| GGCCGTTCCT CTCAATCATC TCTGCCAGAT ACAGCCCTCA CCAGGACGCC GACCCGCTGA | 2580 |
| AGCCCCGGGA GCCGGCCATC ATCAGACACT TCATTGCCTA CAAGAACCAG GACCACGGGG | 2640 |
| CCTGGCTGCG CGGCGGGGAT GTGTGGCTGG ACAGCTGCCA TTTCAGAGGG GAGGCTCAGG | 2700 |
| AAGGCTTCTT GCTTACAGGA ATGAAGGCTG GGGGCATTTT GCTGGGGGGA GATGAGGCAG | 2760 |
| CCTCTGGAAT GGCTCAGGGA TTCAGCCCTC CCTGCCGCTG CCTGCTGAAG CTGGTGACTA | 2820 |
| CGGGGTCGCC CTTTGCTCAC GTCTCTCTGG CCCACTCATG ATGGAGAAGT GTGGTCAGAG | 2880 |
| GGGAGCAATG GGCTTTGCTG CTTATGAGCA CAGAGGAATT CAGTCCCCAG GCAGCCCTGC | 2940 |
| CTCTGACTCC AAGAGGGTGA AGTCCACAGA AGTGAGCTCC TGCCTTAGGG CCTCATTTGC | 3000 |
| TCTTCATCCA GGGAACTGAG CACAGGGGGC CTCCAGGAGA CCCTAGATGT GCTCGTACTC | 3060 |
| CCTCGGCCTG GGATTTCAGA GCTGGAAATA TAGAAAATAT CTAGCCCAAA GCCTTCATTT | 3120 |
| TAACAGATGG GGAAAGTGAG CCCCCAAGAT GGGAAAGAAC CACACAGCTA AGGGAGGGCC | 3180 |
| TGGGGAGCCC CACCCTAGCC CTTGCTGCCA CACCACATTG CCTCAACAAC CGGCCCCAGA | 3240 |
| GTGCCCAGGC ACTCCTGAGG TAGCTTCTGG AAATGGGGAC AAGTCCCCTC GAAGGAAAGG | 3300 |
| AAATGACTAG AGTAGAATGA CAGCTAGCAG ATCTCTTCCC TCCTGCTCCC AGCGCACACA | 3360 |
| AACCCGCCCT CCCCTTGGTG TTGGCGGTCC CTGTGGCCTT CACTTTGTTC ACTACCTGTC | 3420 |
| AGCCCAGCCT GGGTGCACAG TAGCTGCAAC TCCCCATTGG TGCTACCTGG CTCTCCTGTC | 3480 |
| TCTGCAGCTC TACAGGTGAG GCCCAGCAGA GGGAGTAGGG CTCGCCATGT TTCTGGTGAG | 3540 |
| CCAATTTGGC TGATCTTGGG TGTCTGAACA GCTATTGGGT CCACCCCAGT CCCTTTCAGC | 3600 |
| TGCTGCTTAA TGCCCTGCTC TCTCCCTGGC CCACCTTATA GAGAGCCCAA AGAGCTCCTG | 3660 |
| TAAGAGGGAG AACTCTATCT GTGGTTTATA ATCTTGCACG AGGCACCAGA GTCTCCCTGG | 3720 |
| GTCTTGTGAT GAACTACATT TATCCCCTTT CCTGCCCCAA CCACAAACTC TTTCCTTCAA | 3780 |
| AGAGGGCCTG CCTGGCTCCC TCCACCCAAC TGCACCCATG AGACTCGGTC CAAGAGTCCA | 3840 |
| TTCCCCAGGT GGGAGCCAAC TGTCAGGGAG GTCTTTCCCA CCAAACATCT TTCAGCTGCT | 3900 |
| GGGAGGTGAC CATAGGGCTC TGCTTTTAAA GATATGGCTG CTTCAAAGGC CAGAGTCACA | 3960 |
| GGAAGGACTT CTTCCAGGGA GATTAGTGGT GATGGAGAGG AGAGTTAAAA TGACCTCATG | 4020 |
| TCCTTCTTGT CCACGGTTTT GTTGAGTTTT CACTCTTCTA ATGCAAGGGT CTCACACTGT | 4080 |
| GAACCACTTA GGATGTGATC ACTTTCAGGT GGCCAGGAAT GTTGAATGTC TTTGGCTCAG | 4140 |
| TTCATTTAAA AAAGATATCT ATTTGAAAGT TCTCAGAGTT GTACATATGT TTCACAGTAC | 4200 |
| AGGATCTGTA CATAAAAGTT TCTTTCCTAA ACCATTCACC AAGAGCCAAT ATCTAGGCAT | 4260 |
| TTTCTTGGTA GCACAAATTT TCTTATTGCT TAGAAAATTG TCCTCCTTGT TATTTCTGTT | 4320 |
| TGTAAGACTT AAGTGAGTTA GGTCTTTAAG GAAAGCAACG CTCCTCTGAA ATGCTTGTCT | 4380 |
| TTTTTCTGTT GCCGAAATAG CTGGTCCTTT TTCGGGAGTT AGATGTATAG AGTGTTTGTA | 4440 |
| TGTAAACATT TCTTGTAGGC ATCACCATGA ACAAAGATAT ATTTTCTATT TATTTATTAT | 4500 |
| ATGTGCACTT CAAGAAGTCA CTGTCAGAGA AATAAAGAAT TGTCTTAAAT GTCATGATTG | 4560 |
| GAGATGTCCT TTGCATTGCT TGGAAGGGGT GTACCTAGAG CCAAGGAAAT TGGCTCTGGT | 4620 |
| TTGGAAAAAT TTTGCTGTTA TTATAGTAAA CATACAAAGG ATGTCAAAAA AAAAAAAAAA | 4680 |
| AAAAAAAAAA AAAAAAAAAA AA | |

CVA7 variant Protein sequence (SEQ ID NO: 6)

| | |
|---|---|
| Protein Accession #: | Eos sequence |

1 11 21 31 41 51
| | | | | |

| | |
|---|---|
| MGAAGRQDFL FKAMLTISWL TLTCFPGATS TVAAGCPDQS PELQPWNPGH DQDHHVHIGQ | 60 |
| GKTLLLTSSA TVYSIHISEG GKLVIKDHDE PIVLRTRHIL IDNGGELHAG SALCPFQGNF | 120 |
| TIILYGRADE GIQPDPYYGL KYIGVGKGGA LELHGQKKLS WTFLNKTLHP GGMAEGGYFF | 180 |
| ERSWGHRGVI VHVIDPKSGT VIHSDRFDTY RSKKESERLV QYLNAVPDGR ILSVAVNDEG | 240 |
| SRNLDDMARK AMTKLGSKHF LHLGFRHPWS FLTVKGNPSS SVEDHIEYHG HRGSAAARVF | 300 |
| KLFQTEHGEY FNVSLSSEWV QDVEWTEWFD HDKVSQTKGG EKISDLWKAH PGKICNRPID | 360 |
| IQATTMDGVN LSTEVVYKKG QDYRFACYDR GRACRSYRVR FLCGKPVRPK LTVTIDTNVN | 420 |
| STILNLEDNV QSWKPGDTLV IASTDYSMYQ AEEFQVLPCR SCAPNQVKVA GKPMYLHIGE | 480 |
| EIDGVDMRAE VGLLSRNIIV MGEMEDKCYP YRNHICNFFD FDTFGGHIKF ALGFKAAHLE | 540 |
| GTELKHMGQQ LVGQYPIHFH LAGDVDERGG YDPPTYIRDL SIHHTFSRCV TVHGSNGLLI | 600 |
| KDVVGYNSLG HCFFTEDGPE ERNTFDHCLG LLVKSGTLLP SDRDSKMCKM ITEDSYPGYI | 660 |
| PKPRQDCNAV STFWMANPNN NLINCAAAGS EETGFWFIFH HVPTGPSVGM YSPGYSEHIP | 720 |
| LGKFYNNRAH SNYRAGMIID NGVKTTEASA KDKRPFLSII SARYSPHQDA DPLKPREPAI | 780 |
| IRHFIAYKNQ DHGAWLRGGD VWLDSCHFRG EAQEGFLLTG MKAGGILLGG DEAASGMAQG | 840 |
| FSPPCRCLLK LVTTGSPFAH VSLAHS | |

What is claimed is:

1. A method of detecting colorectal cancer in a human individual comprising:
detecting one or more colorectal cancer-associated protein in an extracellular biological sample obtained from a human individual;
wherein the presence of colorectal cancer-associated protein in said extracellular biological sample indicates colorectal cancer in said human individual.

2. The method according to claim 1, wherein said colorectal cancer-associated protein is at least 90% identical to CVA7 or CBF9.

3. The method according to claim 2, wherein said colorectal cancer-association protein is CCA7 or CBF9.

4. A method for detecting the presence of a colorectal cancer-associated protein in an extracelular biological sample, the method comprising contacting the biological sample with a binding agent which specifically binds to a colorectal cancer-associated protein selected from the group consisting of CVA7 and CBF9, thereby detecting the presence of the colorectal cancer-associated protein in the extracellular biological sample.

5. The method of claim 4, wherein the binding agent specifically binds CVA7.

6. The method of claim 4, wherein the binding agent specifically binds CBF9.

7. The method of claim 4, wherein the biological sample is contacted with a first binding agent that specifically binds CVA7 and a second binding agent that specifically binds CBF9.

8. The method of claim 4, wherein the extracellular biological sample is selected from the group consisting of serum, whole blood, plasma, urine, saliva, sputum, tears, and cerebrospinal fluid.

9. The method of claim 8, wherein the extracellular biological sample is blood or serum.

10. The method of claim 4, wherein the binding agent is an antibody.

11. The method of claim 10, wherein the antibody is a monoclonal antibody.

12. The method of claim 10, wherein the antibody is a polyclonal antibody.

13. The method of claim 4, wherein the binding agent is bound to a solid support.

14. The method of claim 13, wherein the solid support comprises nitrocelilgose.

15. The method of claim 13, wherein the solid support is a well of a microtiter plate.

16. The method of claim 4, wherein the binding agent is detectably labled.

17. The method of claim 16, wherein the label is selected from the group consisting of a radiolabel, and a fluorescent label.

18. The method of claim 16, wherein the label is a detectable enzyme. 1

19. The method of claim 18, wherein the detectable enzyme is alkaline phosphatase.

20. A kit for detecting the presence or absence of a colorectal cancer-associated protein in an extracellular biological sample, the kit comprising a binding agent which specifically binds to a colorectal cancer-associated protein selected from the group consisting of CVA7 and CBF9 and assay reagents for detecting the presence or absence of the colorectal cancer-associated protein in the extracellular biological sample.

21. The kit of claim 20, wherein the binding agent is labeled.

22. The kit of claim 20, which comprises a first binding agent that specifically binds CVA7 and a second binding agent at specifically binds CBF9.

23. The kit of claim 20, wherein the binding agent is an antibody.

24. The kit of claim 23, wherein the antibody is a monoclonal antibody or a polyclonal antibody.

25. The kit of claim 20, wherein the binding agent is bound to a solid support.


SEQUENCE LISTING (6 sequences)

SEQ ID NO: 1 - DNA, 3375 bp, Homo sapien
gacagtgttcgcggctgcaccgctcggaggctgggtgacccgcgtagaagtgaagtacttttttatttgcagacctgggccgatgccgctttaaaaaacgcgaggggctctatgcacctccctggcggtagttcctccgacctcagccgggtcgggtcgtgccgccctctcccaggagagacaaacaggtgtcccacgtggcagccgcgccccgggcgcccctcctgtgatcccgtagcgccccctggcccgagccgcgcccgggtctgtgagtagagccgcccgggcaccgagcgctggtcgccgctctccttccgttatatcaacatgccccctttcctgttgctggaggccgtctgtgttttcctgttttccagagtgcccccatctctccctctccaggaagtccatgtaagcaaagaaaccatcgggaagatttcagctgccagcaaaatgatgtggtgctcggctgcagtggacatcatgtttctgttagatgggtctaacagcgtcgggaaagggagctttgaaaggtccaagcactttgccatcacagtctgtgacggtctggacatcagccccgagagggtcagagtgggagcattccagttcagttccactcctcatctggaattccccttggattcattttcaacccaacaggaagtgaaggcaagaatcaagaggatggttttcaaaggagggcgcacggagacggaacttgctctgaaataccttctgcacagagggttgcctggaggcagaaatgcttctgtgccccagatcctcatcatcgtcactgatgggaagtcccagggggatgtggcactgccatccaagcagctgaaggaaaggggtgtcactgtgtttgctgtgggggtcaggtttcccaggtgggaggagctgcatgcactggccagcgagcctagagggcagcacgtgctgttggctgagcaggtggaggatgccaccaacggcctcttcagcaccctcagcagctcggccatctgctccagcgccacgccagactgcagggtcgaggctcacccctgtgagcacaggacgctggagatggtccgggagttcgctggcaatgccccatgctggagaggatcgcggcggacccttgcggtgctggctgcacactgtcccttctacagctggaagagagtgttcctaacccaccctgccacctgctacaggaccacctgcccaggcccctgtgactcgcagccctgccagaatggaggcacatgtgttccagaaggactggacggctaccagtgcctctgcccgctggcctttggaggggaggctaactgtgccctgaagctgagcctggaatgcagggtcgacctcctcttcctgctggacagctctgcgggcaccactctggacggcttcctgcgggccaaagtcttcgtgaagcggtttgtgcgggccgtgctgagcgaggactctcgggcccgagtgggtgtggccacatacagcagggagctgctggtggcggtgcctgtgggggagtaccaggatgtgcctgacctggtctggagcctcgatggcattcccttccgtggtggccccaccctgacgggcagtgccttgcggcaggcggcagagcgtggcttcgggagcgccaccaggacaggccaggaccggccacgtagagtggtggttttgctcactgagtcacactccgaggatgaggttgcgggcccagcgcgtcacgcaagggcgcgagagctgctcctgctgggtgtaggcagtgaggccgtgcgggcagagctggaggagatcacaggcagcccaaagcatgtgatggtctactcggatcctcaggatctgttcaaccaaatccctgagctgcaggggaagctgtgcagccggcagcggccagggtgccggacacaagccctggacctcgtcttcatgttggacacctctgcctcagtagggcccgagaattttgctcagatgcagagctttgtgagaagctgtgccctccagtttgaggtgaaccctgacgtgacacaggtcggcctggtggtgtatggcagccaggtgcagactgccttcgggctggacaccaaacccacccgggctgcgatgctgcgggccattagccaggccccctacctaggtggggtgggctcagccggcaccgccctgctgcacatctatgacaaagtgatgaccgtccagaggggtgcccggcctggtgtccccaaagctgtggtggtgctcacaggcgggagaggcgcagaggatgcagccgttcctgcccagaagctgaggaacaatggcatctctgtcttggtcgtgggcgtggggcctgtcctaagtgagggtctgcggaggcttgcaggtccccgggattccctgatccacgtggcagcttacgccgacctgcggtaccaccaggacgtgctcattgagtggctgtgtggagaagccaagcagccagtcaacctctgcaaacccagcccgtgcatgaatgagggcagctgcgtcctgcagaatgggagctaccgctgcaagtgtcgggatggctgggagggcccccactgcgagaaccgtgagtggagctcttgctctgtatgtgtgagccagggatggattcttgagacgcccctgaggcacatggctcccgtgcaggagggcagcagccgtacccctcccagcaactacagagaaggcctgggcactgaaatggtgcctaccttctggaatgtctgtgccccaggtccttagaatgtctgcttcccgccgtggccaggaccactattctcactgagggaggaggatgtcccaactgcagccatgctgcttagagacaagaaagcagctgatgtcacccacaaacgatgttgttgaaaagttttgatgtgtaagtaaatacccactttctgtacctgctgtgccttgttgaggctatgtcatctgccacctttcccttgaggataaacaaggggtcctgaagacttaaatttagcggcctgacgttcctttgcacacaatcaatgctcgccagaatgttgttgacacagtaatgcccagcagaggcctttactagagcatcctttggacggcgaaggccacggcctttcaagatggaaagcagcagcttttccacttccccagagacattctggatgcatttgcattgagtctgaaagggggcttgagggacgtttgtgacttcttggcgactgccttttgtgtgtggaagagacttggaaaggtctcagactgaatgtgaccaattaaccagcttggttgatgatgggggaggggctgagttgtgcatgggcccaggtctggagggccacgtaaaatcgttctgagtcgtgagcagtgtccaccttgaaggtcttc

SEQ ID NO: 2 - protein, 807 aa, Homo sapien
MPPFLLLEAVCVFLFSRVPPSLPLQEVHVSKETIGKISAASKMMWCSAAVDIMFLLDGSNSVGKGSFERSKHFAITVCDGLDISPERVRVGAFQFSSTPHLEFPLDSFSTQQEVKARIKRMVFKGGRTETELALKYLLHRGLPGGRNASVPQILIIVTDGKSQGDVALPSKQLKERGVTVFAVGVRFPRWEELHALASEPRGQHVLLAEQVEDATNGLFSTLSSSAICSSATPDCRVEAHPCEHRTLEMVREFAGNAPCWRGSRRTLAVLAAHCPFYSWKRVFLTHPATCYRTTCPGPCDSQPCQNGGTCVPEGLDGYQCLCPLAFGGEANCALKLSLECRVDLLFLLDSSAGTTLDGFLRAKVFVKRFVRAVLSEDSRARVGVATYSRELLVAVPVGEYQDVPDLVWSLDGIPFRGGPTLTGSALRQAAERGFGSATRTGQDRPRRVVVLLTESHSEDEVAGPARHARARELLLLGVGSEAVRAELEEITGSPKHVMVYSDPQDLFNQIPELQGKLCSRQRPGCRTQALDLVFMLDTSASVGPENFAQMQSFVRSCALQFEVNPDVTQVGLVVYGSQVQTAFGLDTKPTRAAMLRAISQAPYLGGVGSAGTALLHIYDKVMTVQRGARPGVPKAVVVLTGGRGAEDAAVPAQKLRNNGISVLVVGVGPVLSEGLRRLAGPRDSLIHVAAYADLRYHQDVLIEWLCGEAKQPVNLCKPSPCMNEGSCVLQNGSYRCKCRDGWEGPHCENREWSSCSVCVSQGWILETPLRHMAPVQEGSSRTPPSNYREGLGTEMVPTFWNVCAPGP

SEQ ID NO: 3 - DNA, 5808 bp, Homo sapien
gctcacccaggaaaaatatgcaatcgtcccattgatatacaggccactacaatggatggagttaacctcagcaccgaggttgtctacaaaaaaggccaggattataggtttgcttgctacgaccggggcagagcctgccggagctaccgtgtacggttcctctgtgggaagcctgtgaggcccaaactcacagtcaccattgacaccaatgtgaacagcaccattctgaacttggaggataatgtacagtcatggaaacctggagataccctggtcattgccagtactgattactccatgtaccaggcagaagagttccaggtgcttccctgcagatcctgcgcccccaaccaggtcaaagtggcagggaaaccaatgtacctgcacatcggggaggagatagacggcgtggacatgcgggcggaggttgggcttctgagccggaacatcatagtgatgggggagatggaggacaaatgctacccctacagaaaccacatctgcaatttctttgacttcgatacctttgggggccacatcaagtttgctctgggatttaaggcagcacacttggagggcacggagctgaagcatatgggacagcagctggtgggtcagtacccgattcacttccacctggccggtgatgtagacgaaaggggaggttatgacccacccacatacatcagggacctctccatccatcatacattctctcgctgcgtcacagtccatggctccaatggcttgttgatcaaggacgttgtgggctataactctttgggccactgcttcttcacggaagatgggccggaggaacgcaacacttttgaccactgtcttggcctccttgtcaagtctggaaccctcctcccctcggaccgtgacagcaagatgtgcaagatgatcacaggagactcctacccagggtacatccccaagcccaggcaagactgcaatgctgtgtccaccttctggatggccaatcccaacaacaacctcatcaactgtgccgctgcaggatctgaggaaactggattttggtttatttttcaccacgtaccaacgggcccctccgtgggaatgtactccccaggttattcagagcacattccactgggaaaattctataacaaccgagcacattccaactaccgggctggcatgatcatagacaacggagtcaaaaccaccgaggcctctgccaaggacaagcggccgttcctctcaatcatctctgccagatacagccctcaccaggacgccgacccgctgaagccccgggagccggccatcatcagacacttcattgcctacaagaaccaggaccacggggcctggctgcgcggcggggatgtgtggctggacagctgccggtttgctgacaatggcattggcctgaccctggccagtggtggaaccttcccgtatgacgacggctccaagcaagagataaagaacagcttgtttgttggcgagagtggcaacgtggggacggaaatgatggacaataggatctggggccctggcggcttggaccatagcggaaggaccctccctataggccagaattttccaattagaggaattcagttatatgatggccccatcaacatccaaaactgcactttccgaaagtttgtggccctggagggccggcacaccagcgccctggccttccgcctgaataatgcctggcagagctgcccccataacaacgtgaccggcattgcctttgaggacgttccgattacttccagagtgttcttcggagagcctgggccctggttcaaccagctggacatggatggggataagacatctgtgttccatgacgtcgacggctccgtgtccgagtaccctggctcctacctcacgaagaatgacaactggctggtccggcacccagactgcatcaatgttcccgactggagaggggccatttgcagtgggtgctatgcacagatgtacattcaagcctacaagaccagtaacctgcgaatgaagatcatcaagaatgacttccccagccaccctctttacctggagggggcgctcaccaggagcacccattaccagcaataccaaccggttgtcaccctgcagaagggctacaccatccactgggaccagacggcccccgccgaactcgccatctggctcatcaacttcaacaagggcgactggatccgagtggggctctgctacccgcgaggcaccacattctccatcctctcggatgttcacaatcgcctgctgaagcaaacgtccaagacgggcgtcttcgtgaggaccttgcagatggacaaagtggagcagagctaccctggcaggagccactactactgggacgaggactcagggctgttgttcctgaagctgaaagctcagaacgagagagagaagtttgctttctgctccatgaaaggctgtgagaggataaagattaaagctctgattccaaagaacgcaggcgtcagtgactgcacagccacagcttaccccaagttcaccgagagggctgtcgtagacgtgccgatgcccaagaagctctttggttctcagctgaaaacaaaggaccatttcttggaggtgaagatggagagttccaagcagcacttcttccacctctggaacgacttcgcttacattgaagtggatgggaagaagtaccccagttcggaggatggcatccaggtggtggtgattgacgggaaccaagggcgcgtggtgagccacacgagcttcaggaactccattctgcaaggcataccatggcagcttttcaactatgtggcgaccatccctgacaattccatagtgcttatggcatcaaagggaagatacgtctccagaggcccatggaccagagtgctggaaaagcttggggcagacaggggtctcaagttgaaagagcaaatggcattcgttggcttcaaaggcagcttccggcccatctgggtgacactggacactgaggatcacaaagccaaaatcttccaagttgtgcccatccctgtggtgaagaagaagaagttgtgaggacagctgccgcccggtgccacctcgtggtagactatgacggtgactcttggcagcagaccagtgggggatggctgggtcccccagcccctgccagcagctgcctgggaaggccgtgtttcagccctgatgggccaagggaaggctatcagagaccctggtgctgccacctgcccctactcaagtgtctacctggagcccctggggcggtgctggccaatgctggaaacattcactttcctgcagcctcttgggtgcttctctcctatctgtgcctcttcagtgggggtttggggaccatatcaggagacctgggttgtgctgacagcaaagatccactttggcaggagccctgacccagctaggaggtagtctggagggctggtcattcacagatccccatggtcttcagcagacaagtgagggtggtaaatgtaggagaaagagccttggccttaaggaaatctttactcctgtaagcaagagccaacctcacaggattaggagctggggtagaactggctatccttggggaagaggcaagccctgcctctggccgtgtccacctttcaggagactttgagtggcaggtttggacttggactagatgactctcaaaggcccttttagttctgagattccagaaatctgctgcatttcacatggtacctggaacccaacagttcatggatatccactgatatccatgatgctgggtgccccagcgcacacgggatggagaggtgagaactaatgcctagcttgaggggtctgcagtccagtagggcaggcagtcaggtccatgtgcactgcaatgccaggtggagaaatcacagagaggtaaaatggaggccagtgccatttcagaggggaggctcaggaaggcttcttgcttacaggaatgaaggctgggggcattttgctggggggagatgaggcagcctctggaatggctcagggattcagccctccctgccgctgcctgctgaagctggtgactacggggtcgccctttgctcacgtctctctggcccactcatgatggagaagtgtggtcagaggggagcaatgggctttgctgcttatgagcacagaggaattcagtccccaggcagccctgcctctgactccaagagggtgaagtccacagaagtgagctcctgccttagggcctcatttgctcttcatccagggaactgagcacagggggcctccaggagaccctagatgtgctcgtactccctcggcctgggatttcagagctggaaatatagaaaatatctagcccaaagccttcattttaacagatggggaaagtgagcccccaagatgggaaagaaccacacagctaagggagggcctggggagccccaccctagcccttgctgccacaccacattgcctcaacaaccggccccagagtgcccaggcactcctgaggtagcttctggaaatggggacaagtcccctcgaaggaaaggaaatgactagagtagaatgacagctagcagatctcttccctcctgctcccagcgcacacaaacccgccctccccttggtgttggcggtccctgtggccttcactttgttcactacctgtcagcccagcctgggtgcacagtagctgcaactccccattggtgctacctggctctcctgtctctgcagctctacaggtgaggcccagcagagggagtagggctcgccatgtttctggtgagccaatttggctgatcttgggtgtctgaacagctattgggtccaccccagtccctttcagctgctgcttaatgccctgctctctccctggcccaccttatagagagcccaaagagctcctgtaagagggagaactctatctgtggtttataatcttgcacgaggcaccagagtctccctgggtcttgtgatgaactacatttatcccctttcctgccccaaccacaaactctttccttcaaagagggcctgcctggctccctccacccaactgcacccatgagactcggtccaagagtccattccccaggtgggagccaactgtcagggaggtctttcccaccaaacatctttcagctgctgggaggtgaccatagggctctgcttttaaagatatggctgcttcaaaggccagagtcacaggaaggacttcttccagggagattagtggtgatggagaggagagttaaaatgacctcatgtccttcttgtccacggttttgttgagttttcactcttctaatgcaagggtctcacactgtgaaccacttaggatgtgatcactttcaggtggccaggaatgttgaatgtctttggctcagttcatttaaaaaagatatctatttgaaagttctcagagttgtacatatgtttcacagtacaggatctgtacataaaagtttctttcctaaaccattcaccaagagccaatatctaggcattttcttggtagcacaaattttcttattgcttagaaaattgtcctccttgttatttctgtttgtaagacttaagtgagttaggtctttaaggaaagcaacgctcctctgaaatgcttgtcttttttctgttgccgaaatagctggtcctttttcgggagttagatgtatagagtgtttgtatgtaaacatttcttgtaggcatcaccatgaacaaagatatattttctatttatttattatatgtgcacttcaagaagtcactgtcagagaaataaagaattgtcttaaatgtcaaaaaaaaaaaaaaaaaaaaaaaaaaaaaaaa

SEQ ID NO: 4 - protein, 996 aa, Homo sapien
MDGVNLSTEVVYKKGQDYRFACYDRGRACRSYRVRFLCGKPVRPKLTVTIDTNVNSTILNLEDNVQSWKPGDTLVIASTDYSMYQAEEFQVLPCRSCAPNQVKVAGKPMYLHIGEEIDGVDMRAEVGLLSRNIIVMGEMEDKCYPYRNHICNFFDFDTFGGHIKFALGFKAAHLEGTELKHMGQQLVGQYPIHFHLAGDVDERGGYDPPTYIRDLSIHHTFSRCVTVHGSNGLLIKDVVGYNSLGHCFFTEDGPEERNTFDHCLGLLVKSGTLLPSDRDSKMCKMITGDSYPGYIPKPRQDCNAVSTFWMANPNNNLINCAAAGSEETGFWFIFHHVPTGPSVGMYSPGYSEHIPLGKFYNNRAHSNYRAGMIIDNGVKTTEASAKDKRPFLSIISARYSPHQDADPLKPREPAIIRHFIAYKNQDHGAWLRGGDVWLDSCRFADNGIGLTLASGGTFPYDDGSKQEIKNSLFVGESGNVGTEMMDNRIWGPGGLDHSGRTLPIGQNFPIRGIQLYDGPINIQNCTFRKFVALEGRHTSALAFRLNNAWQSCPHNNVTGIAFEDVPITSRVFFGEPGPWFNQLDMDGDKTSVFHDVDGSVSEYPGSYLTKNDNWLVRHPDCINVPDWRGAICSGCYAQMYIQAYKTSNLRMKIIKNDFPSHPLYLEGALTRSTHYQQYQPVVTLQKGYTIHWDQTAPAELAIWLINFNKGDWIRVGLCYPRGTTFSILSDVHNRLLKQTSKTGVFVRTLQMDKVEQSYPGRSHYYWDEDSGLLFLKLKAQNEREKFAFCSMKGCERIKIKALIPKNAGVSDCTATAYPKFTERAVVDVPMPKKLFGSQLKTKDHFLEVKMESSKQHFFHLWNDFAYIEVDGKKYPSSEDGIQVVVIDGNQGRVVSHTSFRNSILQGIPWQLFNYVATIPDNSIVLMASKGRYVSRGPWTRVLEKLGADRGLKLKEQMAFVGFKGSFRPIWVTLDTEDHKAKIFQVVPIPVVKKKKL

SEQ ID NO: 5 - DNA, 4702 bp, Homo sapien
gagctagcgctcaagcagagcccagcgcggtgctatcggacagagcctggcgagcgcaagcggcgcggggagccagcggggctgagcgcggccagggtctgaacccagatttcccagactagctaccactccgcttgcccacgccccgggagctcgcggcgcctggcggtcagcgaccagacgtccggggccgctgcgctcctggcccgcgaggcgtgacactgtctcggctacagacccagagggagcacactgccaggatgggagctgctgggaggcaggacttcctcttcaaggccatgctgaccatcagctggctcactctgacctgcttccctggggccacatccacagtggctgctgggtgccctgaccagagccctgagttgcaaccctggaaccctggccatgaccaagaccaccatgtgcatatcggccagggcaagacactgctgctcacctcttctgccacggtctattccatccacatctcagagggaggcaagctggtcattaaagaccacgacgagccgattgttttgcgaacccggcacatcctgattgacaacggaggagagctgcatgctgggagtgccctctgccctttccagggcaatttcaccatcattttgtatggaagggctgatgaaggtattcagccggatccttactatggtctgaagtacattggggttggtaaaggaggcgctcttgagttgcatggacagaaaaagctctcctggacatttctgaacaagacccttcacccaggtggcatggcagaaggaggctatttttttgaaaggagctggggccaccgtggagttattgttcatgtcatcgaccccaaatcaggcacagtcatccattctgaccggtttgacacctatagatccaagaaagagagtgaacgtctggtccagtatttgaacgcggtgcccgatggcaggatcctttctgttgcagtgaatgatgaaggttctcgaaatctggatgacatggccaggaaggcgatgaccaaattgggaagcaaacacttcctgcaccttggatttagacacccttggagttttctaactgtgaaaggaaatccatcatcttcagtggaagaccatattgaatatcatggacatcgaggctctgctgctgcccgggtattcaaattgttccagacagagcatggcgaatatttcaatgtttctttgtccagtgagtgggttcaagacgtggagtggacggagtggttcgatcatgataaagtatctcagactaaaggtggggagaaaatttcagacctctggaaagctcacccaggaaaaatatgcaatcgtcccattgatatacaggccactacaatggatggagttaacctcagcaccgaggttgtctacaaaaaaggccaggattataggtttgcttgctacgaccggggcagagcctgccggagctaccgtgtacggttcctctgtgggaagcctgtgaggcccaaactcacagtcaccattgacaccaatgtgaacagcaccattctgaacttggaggataatgtacagtcatggaaacctggagataccctggtcattgccagtactgattactccatgtaccaggcagaagagttccaggtgcttccctgcagatcctgcgcccccaaccaggtcaaagtggcagggaaaccaatgtacctgcacatcggggaggagatagacggcgtggacatgcgggcggaggttgggcttctgagccggaacatcatagtgatgggggagatggaggacaaatgctacccctacagaaaccacatctgcaatttctttgacttcgatacctttgggggccacatcaagtttgctctgggatttaaggcagcacacttggagggcacggagctgaagcatatgggacagcagctggtgggtcagtacccgattcacttccacctggccggtgatgtagacgaaaggggaggttatgacccacccacatacatcagggacctctccatccatcatacattctctcgctgcgtcacagtccatggctccaatggcttgttgatcaaggacgttgtgggctataactctttgggccactgcttcttcacggaagatgggccggaggaacgcaacacttttgaccactgtcttggcctccttgtcaagtctggaaccctcctcccctcggaccgtgacagcaagatgtgcaagatgatcacagaggactcctacccagggtacatccccaagcccaggcaagactgcaatgctgtgtccaccttctggatggccaatcccaacaacaacctcatcaactgtgccgctgcaggatctgaggaaactggattttggtttatttttcaccacgtaccaacgggcccctccgtgggaatgtactccccaggttattcagagcacattccactgggaaaattctataacaaccgagcacattccaactaccgggctggcatgatcatagacaacggagtcaaaaccaccgaggcctctgccaaggacaagcggccgttcctctcaatcatctctgccagatacagccctcaccaggacgccgacccgctgaagccccgggagccggccatcatcagacacttcattgcctacaagaaccaggaccacggggcctggctgcgcggcggggatgtgtggctggacagctgccatttcagaggggaggctcaggaaggcttcttgcttacaggaatgaaggctgggggcattttgctggggggagatgaggcagcctctggaatggctcagggattcagccctccctgccgctgcctgctgaagctggtgactacggggtcgccctttgctcacgtctctctggcccactcatgatggagaagtgtggtcagaggggagcaatgggctttgctgcttatgagcacagaggaattcagtccccaggcagccctgcctctgactccaagagggtgaagtccacagaagtgagctcctgccttagggcctcatttgctcttcatccagggaactgagcacagggggcctccaggagaccctagatgtgctcgtactccctcggcctgggatttcagagctggaaatatagaaaatatctagcccaaagccttcattttaacagatggggaaagtgagcccccaagatgggaaagaaccacacagctaagggagggcctggggagccccaccctagcccttgctgccacaccacattgcctcaacaaccggccccagagtgcccaggcactcctgaggtagcttctggaaatggggacaagtcccctcgaaggaaaggaaatgactagagtagaatgacagctagcagatctcttccctcctgctcccagcgcacacaaacccgccctccccttggtgttggcggtccctgtggccttcactttgttcactacctgtcagcccagcctgggtgcacagtagctgcaactccccattggtgctacctggctctcctgtctctgcagctctacaggtgaggcccagcagagggagtagggctcgccatgtttctggtgagccaatttggctgatcttgggtgtctgaacagctattgggtccaccccagtccctttcagctgctgcttaatgccctgctctctccctggcccaccttatagagagcccaaagagctcctgtaagagggagaactctatctgtggtttataatcttgcacgaggcaccagagtctccctgggtcttgtgatgaactacatttatcccctttcctgccccaaccacaaactctttccttcaaagagggcctgcctggctccctccacccaactgcacccatgagactcggtccaagagtccattccccaggtgggagccaactgtcagggaggtctttcccaccaaacatctttcagctgctgggaggtgaccatagggctctgcttttaaagatatggctgcttcaaaggccagagtcacaggaaggacttcttccagggagattagtggtgatggagaggagagttaaaatgacctcatgtccttcttgtccacggttttgttgagttttcactcttctaatgcaagggtctcacactgtgaaccacttaggatgtgatcactttcaggtggccaggaatgttgaatgtctttggctcagttcatttaaaaaagatatctatttgaaagttctcagagttgtacatatgtttcacagtacaggatctgtacataaaagtttctttcctaaaccattcaccaagagccaatatctaggcattttcttggtagcacaaattttcttattgcttagaaaattgtcctccttgttatttctgtttgtaagacttaagtgagttaggtctttaaggaaagcaacgctcctctgaaatgcttgtcttttttctgttgccgaaatagctggtcctttttcgggagttagatgtatagagtgtttgtatgtaaacatttcttgtaggcatcaccatgaacaaagatatattttctatttatttattatatgtgcacttcaagaagtcactgtcagagaaataaagaattgtcttaaatgtcatgattggagatgtcctttgcattgcttggaaggggtgtacctagagccaaggaaattggctctggtttggaaaaattttgctgttattatagtaaacatacaaaggatgtcaaaaaaaaaaaaaaaaaaaaaaaaaaaaaaaaaaaaa

SEQ ID NO: 6 - protein, 866 aa, Homo sapien
MGAAGRQDFLFKAMLTISWLTLTCFPGATSTVAAGCPDQSPELQPWNPGHDQDHHVHIGQGKTLLLTSSATVYSIHISEGGKLVIKDHDEPIVLRTRHILIDNGGELHAGSALCPFQGNFTIILYGRADEGIQPDPYYGLKYIGVGKGGALELHGQKKLSWTFLNKTLHPGGMAEGGYFFERSWGHRGVIVHVIDPKSGTVIHSDRFDTYRSKKESERLVQYLNAVPDGRILSVAVNDEGSRNLDDMARKAMTKLGSKHFLHLGFRHPWSFLTVKGNPSSSVEDHIEYHGHRGSAAARVFKLFQTEHGEYFNVSLSSEWVQDVEWTEWFDHDKVSQTKGGEKISDLWKAHPGKICNRPIDIQATTMDGVNLSTEVVYKKGQDYRFACYDRGRACRSYRVRFLCGKPVRPKLTVTIDTNVNSTILNLEDNVQSWKPGDTLVIASTDYSMYQAEEFQVLPCRSCAPNQVKVAGKPMYLHIGEEIDGVDMRAEVGLLSRNIIVMGEMEDKCYPYRNHICNFFDFDTFGGHIKFALGFKAAHLEGTELKHMGQQLVGQYPIHFHLAGDVDERGGYDPPTYIRDLSIHHTFSRCVTVHGSNGLLIKDVVGYNSLGHCFFTEDGPEERNTFDHCLGLLVKSGTLLPSDRDSKMCKMITEDSYPGYIPKPRQDCNAVSTFWMANPNNNLINCAAAGSEETGFWFIFHHVPTGPSVGMYSPGYSEHIPLGKFYNNRAHSNYRAGMIIDNGVKTTEASAKDKRPFLSIISARYSPHQDADPLKPREPAIIRHFIAYKNQDHGAWLRGGDVWLDSCHFRGEAQEGFLLTGMKAGGILLGGDEAASGMAQGFSPPCRCLLKLVTTGSPFAHVSLAHS